(19) United States
(12) Patent Application Publication
(10) Pub. No.: US 2022/0196658 A1
(43) Pub. Date: Jun. 23, 2022

(54) ANTI-VIRUS IgG3 BINDING ASSAY TO ASSESS VIRUS NEUTRALIZATION AND RELATED METHODS

(71) Applicants: University of Washington, Seattle, WA (US); Seattle Children's Hospital d/b/a Seattle Children's Research Institute, Seattle, WA (US)

(72) Inventors: Jennifer Rathe, Seattle, WA (US); Michael J. Gale, JR., Seatle, WA (US)

(21) Appl. No.: 17/558,244

(22) Filed: Dec. 21, 2021

Related U.S. Application Data
(60) Provisional application No. 63/129,704, filed on Dec. 23, 2020.

(51) Int. Cl. G01N 33/569; G01N 33/566; G01N 33/50

(52) U.S. Cl. CPC G01N 33/56983; G01N 2333/165; G01N 33/5091; G01N 33/566

(57) ABSTRACT

The disclosure provides method, kits, and devices for detecting and/or assessing neutralizing antibodies in a sample, e.g. a biological sample. The detection comprises contacting the sample to an antigen of a virus of interest, and detecting the binding of the one or more IgG3 antibodies to the antigen. The presence of IgG3 antibodies that specifically bind the virus antigen indicates neutralizing antibodies against the virus. The detection protocol can be implemented in a variety of configurations and formats, including ELISA-type and lateral flow formats. The method can be used to assess, e.g., a subject's relative immunity or infection status regarding a particular virus, such as SARS-CoV-2.

### CROSS-REFERENCE TO RELATED APPLICATION

This application claims the benefit of Provisional Application No. 63/129,704, filed Dec. 23, 2020, the disclosure of which is incorporated herein by reference in its entirety.

### STATEMENT OF GOVERNMENT LICENSE RIGHTS

This invention was made with Government support under Grant No. AI145296, awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 3915-P1234USUW_Seq_List_FINAL_20211221_ST25.txt. The text file is 48 KB; was created on Dec. 21, 2021; and is being submitted via EFS-Web with the filing of the specification.

### BACKGROUND

SARS-CoV-2 has infected millions of people globally. Even with evolving vaccine technologies, management and prevention of the pandemic requires accurate assessment of immunological status and infection history within the population. Several serological assays to detect SARS-CoV-2 antibodies have been developed but their utility is hindered by limited sensitivity and specificity, and unclear correlation with viral neutralization. Current interpretation of antibody test results is difficult given that individuals with virus-specific antibodies may not exhibit neutralization activity. False positives from any single assay also confound clinical interpretation of serologic test results, warranting coupled analyses of virus-specific antibody titer and virus neutralization activity.

For other well-studied viral respiratory infections and associated vaccine efficacy, established threshold antibody titers correlate with protective immunity from symptomatic infection and this relationship is likely to hold true for SARS-CoV-2. The majority of SARS-CoV-2 infected individuals produce neutralizing antibodies. There have been rare cases of re-infection described despite more than a year of SARS-CoV-2 outbreaks and spread across the world. The re-infection cases lacked assessment of SARS-CoV-2 specific immunity prior to reinfection in that antibody titers and SARS-CoV-2 specific lymphocyte responses were not measured. While the presence of neutralizing antibodies against SARS-CoV-2 may serve as a correlate of protective immunity, the longevity and threshold titer level of protective antibodies is unknown.

A critical problem facing assessment of immune correlates of protection against SARS-CoV-2 is consensus on an accurate, high throughput testing strategy. Comparisons across antibody testing platforms reveal that no single test performs with 100% sensitivity and specificity. Further, no single test has consistently predicted viral neutralization with the detection of any particular anti-SARS-CoV-2 antibody type. These less than ideal test characteristics likely reflect a time-dependent decrease in the correlative relationship between antibody levels and the strength of viral neutralization. Antibody test platforms differ in the antibody isotypes detected (IgG, IgM, IgA, etc.). Further, the immune system production of isotypes is time-dependent and is variably related to viral neutralization. An accurate and consistent SARS-CoV-2 antibody-testing platform has not been identified even when using ideal, control populations. Few studies have evaluated test performance in populations where past infection status and time from infection are unknown nor have such cohorts undergone coupled evaluation of antibody titer and viral neutralization activity. Evaluation of both control and unknown populations in a comprehensive manner is needed to determine an accurate, consistent testing strategy to identify individuals with correlates of viral neutralization and protective immunity to SARS-CoV-2.

Accordingly, a need remains for a facile, sensitive test that accurately indicates immunological status of the subject for a viral infection. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of detecting antibodies in a sample (e.g., a biological sample). The antibodies are effective to neutralize a virus (i.e., “neutralizing antibodies). The method comprises contacting the biological sample to a virus antigen, and detecting the binding of one or more IgG3 antibodies to the antigen. Binding of one or more IgG3 antibodies to the antigen indicates neutralizing antibodies in the biological sample.

In some embodiments, the virus is characterized as a respiratory virus for a subject. In some embodiments, the virus is a coronavirus. In some embodiments, the coronavirus is characterized as being in the subfamily Orthocoronavirinae. In some embodiments, the coronavirus causes the common cold, Middle East respiratory syndrome (MERS-CoV), severe acute respiratory syndrome (SARS), or coronavirus disease 2019 (COVID-19). In some embodiments, the coronavirus is severe acute respiratory syndrome coronavirus 2 (SARS-CoV-2). In some embodiments, the virus antigen is SARS-CoV-2 spike protein, or a portion thereof.

In some embodiments, the sample is a biological sample that is obtained from a subject. In some embodiments, the presence of IgG3 antibodies indicates the subject has neutralizing antibodies against the virus.

In some embodiments, the virus antigen is immobilized on a solid substrate. In some embodiments, detecting the binding of one or more IgG3 antibodies to the antigen comprises contacting the biological sample with an affinity reagent specific for IgG3 antibody. In some embodiments, the affinity reagent is coupled directly or indirectly to a detectable moiety. In some embodiments, the contacting and detecting are performed in an ELISA format or lateral flow format. In some embodiments, the method further comprises quantifying the IgG3 antibodies that bind to the antigen to provide an IgG3 level, and comparing the IgG3 level to a reference standard.

In some embodiments, the biological sample is or comprises blood, serum, plasma, sputum, nasopharyngeal swab specimen, buccal or oral swab specimen, cerebral spinal fluid, rectal swab or stool specimen, or is derived therefrom.

In another aspect, the disclosure provides a method of determining whether a subject has neutralizing antibodies to a virus of the subfamily Orthocoronavirinae. The method comprises contacting a sample obtained from the subject with a virus antigen and detecting binding of one or more IgG3 antibodies to the virus antigen. Detection of binding one or more IgG3 antibodies to the virus antigen indicates that the subject has neutralizing antibodies to the virus. Alternatively, lack of detection of binding one or more IgG3 antibodies to the virus antigen indicates that the subject lacks neutralizing antibodies to the virus.

In some embodiments, the virus is a coronavirus that causes the common cold, Middle East respiratory syndrome coronavirus (MERS-CoV), severe acute respiratory syndrome (SARS), or coronavirus disease 2019 (COVID-19). In some embodiments, the coronavirus is severe acute respiratory syndrome coronavirus 2 (SARS-CoV-2). In some embodiments, the virus antigen is SARS-CoV-2 spike protein, or a portion thereof. In some embodiments, the virus antigen is immobilized on a solid substrate.

In some embodiments, the detecting the binding of one or more IgG3 antibodies to the antigen comprises contacting the sample with an affinity reagent specific for IgG3 antibody.

In some embodiments, the affinity reagent is coupled directly or indirectly to a detectable moiety. In some embodiments, the contacting and detecting are performed in an ELISA format or lateral flow format.

In some embodiments, the presence of neutralizing antibodies indicates that the subject has been infected with or immunized against the virus resulting in at least partial protection against disease caused by the virus. In some embodiments, the method further comprises quantifying the IgG3 antibodies that bind to the antigen to provide an IgG3 level, and comparing the IgG3 level to a reference standard. In some embodiments, when the IgG3 level is equal to or exceeds the reference standard the subject is determined to have been infected with or immunized against the virus resulting in at least partial protection against disease caused by the virus. In some embodiments, the lack of neutralizing antibodies or lack of sufficient levels of neutralizing antibodies compared to a reference standard indicates that the subject has insufficient immunity against the virus and the method further comprises administering a vaccine or vaccine booster against the virus.

In some embodiments, the sample is or comprises blood, serum, plasma, sputum, nasopharyngeal swab specimen, buccal or oral swab specimen, or is derived therefrom.

In another aspect, the disclosure provides a kit. The comprises a virus antigen and an affinity reagent that specifically binds to IgG3 antibody. In some embodiments, the virus antigen is an antigen from a virus of the subfamily Orthocoronavirinae. In some embodiments, the virus is a coronavirus that causes the common cold, Middle East respiratory syndrome coronavirus (MERS-CoV), severe acute respiratory syndrome (SARS), or coronavirus disease 2019 (COVID-19). In some embodiments, the coronavirus is severe acute respiratory syndrome coronavirus 2 (SARS-CoV-2). In some embodiments, the virus antigen is SARS-CoV-2 spike protein, or a fragment thereof. In some embodiments, the virus antigen is immobilized on a solid substrate. In some embodiments, the virus antigen is coupled directly or indirectly to a detectable moiety. In some embodiments, the affinity reagent is immobilized on a solid substrate. In some embodiments, the affinity reagent is coupled directly or indirectly to a detectable moiety. In some embodiments, the kit further comprises an assay device configured to receive a biological sample and allow any antibodies present in the biological sample to contact the virus antigen and affinity reagent. In some embodiments, the device is or comprises an ELISA plate. In some embodiments, the device is or comprises an lateral flow strip.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B graphically illustrate a comparison of ELISA IgG platforms. (1A) Positive and negative control serum sample total IgG antibody recognition of SARS-CoV-2 RBD, spike protein, and UV-inactivated SARS-CoV-2. (1B) Unknown serum samples total IgG RBD and spike assays. Mean+/−SD, Student's t-tests for comparisons of mean of groups. Dotted lines represent cut-off of 3SD from the mean of the negative control samples to designate ‘positive’ antibody samples.

FIGS. 2A-2C graphically illustrate a comparison of ELISA antibody isotype performance. (2A) IgM antibody recognition of SARS-CoV-2 RBD and spike proteins of all serum samples. (2B) IgG1, IgG3, and IgA antibody recognition of spike protein of all samples. Mean+/−SD, Student's t-tests for comparisons of mean of groups. Dotted line represents cut-off of 3SD from the mean of the negative control samples to designate ‘positive’ antibody samples. (2C) IgG SARS-CoV-2 Abbott NP chemiluminescent microparticle immunoassay results, assay quantitative measurement output, >1.39=positive result.

FIGS. 3A and 3B graphically illustrate plaque reduction neutralization test (PRNT) results. (3A) Linear regression analyses correlating magnitude of neutralization PRNT₈₀ to magnitude of ELISA serum: spike IgG, RBG IgG, NP IgG, and spike IgG3. (3B) PRNT₈₀ results for all samples and for those meeting neutralization criteria. Mean+/−SD, Student's t-tests for comparisons of mean of two groups and ANOVA analyses for difference among means of three groups.

FIGS. 4A and 4B are tables that summarize ELISA and PRNT testing performance across all platforms in the setting of unknown SARS-CoV-2 exposure individuals (N=20). (4A) All of the ELISA platforms are compared based on ELISA assay detection and correlation with detectable neutralization via PRNT. (4B) Comprehensive comparison of the three assays best at detecting antibodies with neutralizing activity. Solid color blocks denote positive ELISA group B subject; solid color+crosshatch denote positive ELISA, SARS-CoV-2 group A subject; PRNT % agreement denotes number of ELISA positives in agreement with PRNT positives. MI denotes misidentified outcomes in which incongruent ELISA results with neutralization detection are not encompassed by the % PRNT agreement calculation.

FIG. 5 is a table demonstrating an assessment of coupled, sequential antibody tests to increase performance. Testing performance was assessed for four possible combinations of ELISA platforms to increase the ability to detect and predict neutralizing antibodies. All the coupled combinations were compared to spike IgG3 as the best performing single ELISA platform. Solid color blocks denote positive ELISA group B subject; solid color+crosshatch denotes positive ELISA, SARS-CoV-2 group A subject; PRNT % agreement denotes number of ELISA positives in agreement with PRNT positives. MI denotes misidentified outcomes in which incongruent ELISA results with neutralization detection were not encompassed by the % PRNT agreement calculation.

FIGS. 6A and 6B provide a study subject description and serologic prevalence estimates. (6A) A map of Seattle with circles denoting the zip codes of the subjects involved in the study. Underlying map colors represent the population density for the areas shown. (6B) Boxes demonstrate the number of subjects, exposure status, and reported symptoms for positive subjects. Group A and B unknown subjects have never been diagnosed with SARS-CoV-2 infection. Group A subjects had close contact with a known infected SARS-CoV-2 individual. Group B subjects had no known exposure to SARS-CoV-2 infected individuals. *Subjects reported symptoms within 5 days of exposure to SARS-CoV-2 positive individuals. **Subjects reported symptoms over the possible exposure window in the Seattle area (Jan. 21, 2020-Apr. 15, 2020). The estimated prevalence of neutralizing SARS-CoV-2 antibodies in the greater Seattle area is 0.035 (3.5%) with associated 95% confidence interval of 0.013-0.073 (1.3%-7.3%). The p value calculated from a comparison of the exposed versus unexposed groups proportion of positive test results in the study was P<0.00001.

FIG. 7 is a flow chart demonstrating the cut off criteria for assessing plaque neutralization reduction test (PRNT), as used in a study described herein.

FIGS. 8A-8C are a series of histograms for age for two cohorts assessed in the present disclosure. The counts are presented for the cohorts separately and in combination.

### DETAILED DESCRIPTION

This disclosure is based on efforts to determine how serologic antibody testing outcome links with virus neutralization of SARS-CoV-2. As described in more detail below, the inventors evaluated a unique set of individuals for SARS-CoV-2 antibody level and viral neutralization. Briefly, serum Ig levels were compared across platforms of viral antigens and antibodies with 15 positive and 30 negative SARS-CoV-2 controls followed by viral neutralization assessment. These platforms were then applied to a clinically relevant cohort of 114 individuals with unknown histories of SARS-CoV-2 infection. In controls, the best performing virus-specific antibody detection platforms were SARS-CoV-2 receptor binding domain (RBD) IgG [sensitivity 87%, specificity 100%, positive predictive value (PPV) 100%, negative predictive value (NPV) 94%], spike IgG3 [sensitivity 93%, specificity 97%, PPV 93%, NPV 97%], and nucleocapsid protein (NP) IgG [sensitivity 93%, specificity 97%, PPV 93%, NPV 97%]. Neutralization of positive and negative control sera showed 100% agreement. Twenty unknown individuals had detectable SARS-CoV-2 antibodies with 16 demonstrating virus neutralization. Spike IgG3 provided the highest accuracy for predicting serologically positive individuals with virus neutralization activity. This data demonstrates that a spike IgG3 antibody test is optimal to categorize patients for correlates of SARS-CoV-2 immune protection status.

In accordance with the foregoing, in one aspect the disclosure provides a method of determining whether a sample, e.g., biological sample, has neutralizing antibodies against a pathogen, e.g., a viral pathogen. Neutralizing antibodies are antibodies that, in addition to simply binding the target pathogen (e.g., virus), when present in vivo reduce the impact of the target pathogen on a subject infected or exposed to the pathogen. Such a reduced impact can be realized in conjunction with other elements of the subject's immune system within the context of the host's immune system. The reduced impact can manifest as resistance to infection, reduced viral load, faster clearance of infection, reduced symptoms as a result of infection, and the like. Further neutralization can be assessed or confirmed in vitro using assays known in the art. Illustrative, non-limiting examples include assays such as focus reduction neutralization test (FRNT) or plaque reduction neutralization test (PRNT), as described in more detail below. The sample can be a biological sample obtained from a subject. In this scenario, any detection of neutralizing antibodies detected in the sample will indicate that the subject has neutralizing antibodies against the virus and, thus, informs the immunological status of the subject against the pathogen. For example, detection of neutralization antibodies in the sample can reveal whether the subject has previously been infected with the virus, whether the subject has been immunized against the virus, and/or the presence or degree to which the subject has manifested an effective immune response against the virus or vaccine composition. This, in turn, informs the degree of potential resistance the subject may have against future infection and, thus, can inform prospective treatment and vaccination strategies and overall risk assessment.

The inventors have demonstrated that the presence of IgG3 antibodies, independent of other antibody isotypes or subclasses, is highly correlated with neutralizing functionality against viruses, e.g., coronavirus. IgG3 antibodies are a subclass of the IgG isotype of antibody. Thus, in this aspect, the method comprises contacting the sample, e.g., the biological sample, to an antigen, e.g., viral antigen, and detecting the binding of one or more IgG3 antibodies to the antigen. Binding of one or more IgG3 antibodies to the antigen indicates neutralizing antibodies in the biological sample.

The presence of IgG3 can be indicative of neutralizing antibody functionality for a variety of pathogens, such as viruses, including rapidly evolving or mutating pathogen strains. In some embodiments the virus is a respiratory virus. In humans, exemplary non-limiting respiratory viruses encompassed by the disclosure include adenoviruses (ADV), human respiratory syncytial virus (HRSV), human bocavirus (HBoV), coronavirus (CoV) including CoV associated with severe acute respiratory syndrome (SARS) (SARS-CoV), human metapneumovirus (HMPV), human parainfluenza virus (HPIV), human respiratory syncytial virus (HRSV), and human rhinovirus (HRV) pharyngoconjunctival fever (PCF). See, e.g., Boncristiani H F, et al. Respiratory Viruses. Encyclopedia of Microbiology. 500-518 (2009). doi: 10.1016/B978-012373944-5.00314-X

In some embodiments, the virus is a coronavirus. Coronaviruses are RNA viruses typified by large, roughly spherical particles. The particle size is highly variable, but typically fall in the average diameter of about 80 to 120 nm (with wider known ranges extending from about 50 to 200 nm in diameter). The particles have a lipid bilayer envelope in which membrane, envelope, and spike proteins are embedded and which provide for particle stability and host cell interaction. The particles contain a positive-sense, single-stranded RNA genome, typically ranging from 26.5 to about 31.1 Kb, which is rather large relative to other viruses.

The coronavirus can be characterized as being in the subfamily Orthocoronavirinae. This subfamily is classified as having four genera: Alphacoronavirus, Betacoronavirus, Gammacoronavirus and Deltacoronavirus. Exemplary species in the genus Alphacoronavirus, include: Alphacoronavirus 1 (TGEV, Feline coronavirus, Canine coronavirus), Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, and Scotophilus bat coronavirus 512. Exemplary species in the genus Betacoronavirus, include: Betacoronavirus 1 (Bovine Coronavirus, Human coronavirus OC43), Hedgehog coronavirus 1, Human coronavirus HKU1, Middle East respiratory syndrome-related coronavirus, Murine coronavirus, Pipistrellus bat coronavirus HKU5, Rousettus bat coronavirus HKU9, Severe acute respiratory syndrome-related coronavirus (SARS-CoV, SARS-CoV-2), and Tylonycteris bat coronavirus HKU4. Exemplary species in the genus Gammacoronavirus, include: Avian coronavirus, and Beluga whale coronavirus SW1. Exemplary species in the genus Deltacoronavirus, include: Bulbul coronavirus HKU11, and Porcine coronavirus HKU15.

In some embodiments, the subject coronavirus causes the common cold. Illustrative, non-limiting examples include human coronaviruses HCoV-OC43, HCoV-HKU1, HCoV-229E, and HCoV-NL63, or variants or strains thereof. In some embodiments, the coronavirus causes Middle East respiratory syndrome (MERS), e.g., Middle East respiratory syndrome coronavirus (MERS-CoV), or variants or strains thereof. In some embodiments, the coronavirus causes severe acute respiratory syndrome (SARS), e.g., SARS coronavirus (SARS-CoV), or variants or strains thereof. In some embodiments, the coronavirus causes coronavirus disease 2019 (COVID-19), e.g., Severe acute respiratory syndrome coronavirus 2 (SARS-CoV-2). Exemplary known strains of the SARS-CoV-2 virus include B.1.1.7 (“alpha”), B.1.351 (“beta”), P.1 (“gamma”), B.1.617.2 (“delta”), B.1.1.52 (“omicron”), B.1.429, B.1.427, and CAL.20C (“epsilon”), P.2 (“zeta”), B.1.525 (“eta”), P.3 (“theta”), B.1.526 (“Iota”), B.1.617.1 (“kappa”), although other variants and lineages exist and are known. All such variants, in addition to variants yet undiscovered or yet to emerge, are encompassed by the present disclosure.

As indicated above, the method of this aspect comprises contacting the biological sample with (or to) a virus antigen. The virus antigen is preferably an antigen that is accessible to antibodies outside the viral particle, e.g., when situated in the blood or extracellular space. Accordingly, the antigen can be a protein that is embedded in the out layer (e.g., capsid, wall, or envelope) of the viral particle. Appropriate antigens can be readily selected by persons of ordinary skill in the art depending on the choice of virus. For example, in the context of the SARS-CoV-2 virus, the antigen can be the spike protein, envelope protein, membrane protein, or nucleocapsid protein, or an immunogenic fragment thereof.

In an exemplary embodiment, the antigen is the SARS-CoV-2 virus spike protein, or an immunogenic fragment thereof (e.g., including a portion of the spike protein that extends out of the envelope into the extra-particle space. For example, in a coronavirus the spike protein is a homotrimeric structure on the surface of the viral membrane. the spike protein is translated as a large polypeptide that is subsequently cleaved to the distal S1 domain, which is responsible for receptor binding, and the membrane-anchored S2 domain, which is responsible for membrane fusion. The SARS-CoV S1 subunit is composed of two distinct domains: an N-terminal domain (S1 NTD) and a receptor-binding domain (S1 RBD), which is also referred to S1 CTD or domain B. Exemplary spike protein polypeptide sequences are described in Genbank entries QHD43416.1, QIZ97039.1, QXX38122.1, QSL77531.1, and the like, incorporated herein by reference in their entireties. Representative spike protein polypeptides comprising sequences of useful antigens are set forth as SEQ ID NOS:1-4. In some embodiments, the antigen comprises an amino acid sequence with at least about 80%, 85%, 90%, 95%, 98%, 99% sequence identity to a consecutive sequence of at least 20, 25, 40, 35, 40, 45, 50, 55, 60, 65, 70, 75, 80, 85, 90, 95, 100, 125, 150, 175, 200, or more amino acids of one of SEQ ID NOS:1-4. In some embodiments, the antigen comprises an amino acid sequence with at least about 65%, 70%, 75%, 80%, 85%, 90%, 95%, 98%, 99% sequence identity to an S1 domain as contained in one of SEQ ID NOS:1-4. In some embodiments, the antigen comprises an amino acid sequence with at least about 65%, 70%, 75%, 80%, 85%, 90%, 95%, 98%, 99% sequence identity to an S2 domain as contained in one of SEQ ID NOS:1-4.

As indicated, the method also comprised detecting specifically the binding of IgG3 subclass antibodies to the virus antigen. Immunoglobulin G (IgG) is a type of antibody that, in humans, represents approximately 75% of the serum antibodies. The canonical IgG antibody has a molecular weight of about 150 kDa and is made of four peptide chains: two gamma heavy chains and two light chains. The heavy chains are linked to each other by a disulfide bond. IgG3 is the third (of four) most prevalent subtypes of IgG total. IgG3 antibodies have variations in the Fc region that serves as a high activator of complement and has high affinity for Fc receptors on phagocytic cells. IgG3 antibodies are notable for the relatively short half-life compared to other subtypes. IgG3 antibodies can be identified by their unique Fc sequence characteristics. An exemplary IgG3 sequence of an IGHG3 immunoglobulin heavy constant gamma 3 is encoded by the gene identified as Genbank Gene ID 3502. A representative protein sequence for the IgG3 heavy chain constant domain comprises the protein sequence set forth in SEQ ID NO:5. Sequence variation within identifiable IgG3 sequences is known and identifiable. As such variants are encompassed by this disclosure. See, e.g., Vidarsson, G., et al., IgG subclasses and allotypes: from structure to effector functions, Front. Immunol., 5:520 (2014; and Chu, T. H., et al. Coming together at the hinges: Therapeutic prospects of IgG3, mAbs, 13(1): 1882028 Taylor & Francis (2021), each of which is incorporated herein by reference in its entirety. In some embodiments, the IgG3 antibodies comprise an amino acid sequence with at least about 85%, 90%, 95%, 98%, 99% sequence identity to a consecutive sequence of at least 20, 25, 40, 35, 40, 45, 50, 55, 60, 65, 70, 75, 80, 85, 90, 95, 100, 125, 150, 175, 200, 225, 250, 275, 300 or more amino acids of SEQ ID NO:5.

In some embodiments, detection of IgG3 antibodies can be performed with an immune assay that incorporates contacting the sample with an affinity reagent specific for IgG3. The term “affinity reagent” as used in this context is described in more detail below. Generally, the affinity reagent in this context can include antibodies, antibody-like molecules (e.g., antibody fragments or derivatives), aptamers, and the like, that are selected for specific binding to IgG3. In this context, specific binding to IgG3 indicates that the affinity reagent binds selectively to IgG3 subclass to the exclusion or substantial exclusion of other IgG subclasses (i.e., IgG1, IgG2, IgG4) and other antibodies. Thus, the affinity reagent preferably does not bind to, or substantially bind to, other antibody isotypes or other subclasses if IgG under standard conditions. Examples of such conditions are provided below and can be readily determined by skilled practitioners. Exemplary affinity reagents that specifically bind IgG3 are commercially available. For example, murine anti-human IgG3 antibodies (SouthernBiotech, 9210) were used in ELISA studies, as described in Example 1.

The affinity reagent can, depending on the assay format, be coupled directly or indirectly to a detectable moiety or label. A detectable moiety or label, as used herein, refers to a moiety attached to an affinity reagent or other reagent to render the reaction between the affinity reagent and the analyte detectable. The reagent (e.g., affinity reagent) so labeled is referred to as being “detectably labeled.”

Many detectable labels are known that can be readily attached, covalently or non-covalently, to the affinity reagent. A label can produce a signal itself that is detectable by visual or instrumental means. Various labels include signal-producing substances, luminescent moieties, bioluminescent moieties, radioactive moieties, positron emitting metals, nonradioactive paramagnetic metal ions, and the like. A nonlimiting example of a luminescent moiety includes luminol. Non-limiting examples of bioluminescent moieties include luciferase, luciferin, and aequorin. Other fluorescent or luminescent proteins include green fluorescent protein (GFP), yellow fluorescence protein (YFP), red fluorescent protein (RFP), and the like. Nonlimiting examples of photo-activatable proteins such as photoactivatable (PA)-green fluorescent protein (GFP), PA-mCherry, PA-mRFP1, PS-CFP2, mEos, tdEos, Kaede, KikGr, mKiGR, derivatives thereof and the like. Nonlimiting photo-activatable proteins encompassed by the present disclosure include reversible photo-activatable proteins such as photoactivatable Dronpa, Padron, rsCherry, rsCherryrev, and FP595, derivatives thereof, and the like. Additional examples of photo-activatable proteins are known and are encompassed by this disclosure. See, e.g., Fluorescent Proteins 101: A Desktop Resource (1st Edition). Tyler J. Ford and The Addgene Team I October, 2017, and references cited therein, each of which is incorporated herein by reference in its entirety. Nonlimiting examples of suitable radioactive moieties include a radioactive metal ion, e.g., alpha-emitters or other radioisotopes such as, for example, iodine (1311, 1251, 1231, 121I), carbon (14C), sulfur (35S), tritium (3H), indium (115mIn, 113mIn, 112In, U1In), and technetium (99Tc, 99mTc), thallium (201Ti), gallium (68Ga, /18t7\ 153r WO 2016/073853-44-PCT/US2015/059468 67Ga), palladium (103Pd), molybdenum (99Mo), xenon (133Xe), fluorine (10F), 13JSm, Lu, 159Gd, 149Pm, 140La, 175Yb, 166Ho, 90Y, 47Sc, 86R, 188Re, 142Pr, 105Rh, 97Ru, 68Ge, 57Co, 65Zn, 85Sr, 32P, 153Gd, 169Yb, 51Cr, 54Mn, 75Se, and tin (113Sn, 117Sn). Furthermore, the detectable label can simply be colored particles, such as latex or metallic beads (e.g., gold beads).

A label can also be a moiety that does not itself emit a signal but can be detected upon its activity with a substrate. For example, the label can be a suitable enzyme, such as horseradish peroxidase, alkaline phosphatase, β-galactosidase, glucose oxidase, or acetylcholinesterase, that can facilitate a detectable signal under specifically applied conditions using known substrates.

The detectable moieties or labels can be coupled or conjugated either directly to the affinity reagents of the disclosure or indirectly, through an intermediate (such as, for example, a linker) using suitable techniques. Furthermore, the concept of indirect coupling can also include use of secondary (and even tertiary, etc.) affinity reagents that are themselves coupled directly (or indirectly) to the detectable moiety or label, where the secondary affinity reagent binds to the affinity reagent that is bound to the antigen in a manner that does not interfere with the primary affinity reagent binding to the antigen (or the tertiary affinity reagent binds to the secondary affinity reagent, which binds to the primary affinity reagent, and so on.)

A person of ordinary skill in the art will readily appreciate that the disclosed method can be performed in a variety of assay formats, all of which are encompassed by this disclosure.

In some embodiments, assays rely on a combination of reagents that serve as capture reagents (i.e., to immobilize the target, here the antigen-specific IgG3 antibodies) and detection reagents (i.e., to facilitate, directly or indirectly, the detectable signal indicating captured IgG3 antibodies). The antigen itself can serve as either the capture reagent or detection reagent. Thus, the antigen can be immobilized or not immobilized, depending on the format. In some embodiments, the IgG3-specific affinity reagent described above can serve as either the capture reagent or the detection reagent as appropriate and in coordination with the antigen in its role as a capture reagent or a detection reagent.

For example, the assay can be performed on a plate (e.g., ELISA plate) where the sample and various reagents are mutually contacted. For example, in one detection format, the antigen is immobilized to the solid surface of an assay plate. The sample is contacted to the surface and any antigen-specific IgG3 antibodies that are present are allowed to bind to the immobilized antigen, thereby becoming immobilized. The presence of the bound IgG3 antibodies can be detected, e.g., by use of an affinity reagent specific for IgG3 antibodies. The affinity reagent can itself be detectably labeled (i.e., for direct detection), as described above, or secondary (or secondary plus tertiary, etc.) affinity reagents can be contacted to the solid surface where the secondary (or tertiary, etc.) affinity reagents are detectably labeled (i.e., for indirect detection). Strategies utilizing secondary or even additional affinity reagents to implement indirect detection can be advantageous to amplify the amount of detectable signal corresponding with each IgG3 antibody that is bound, thus increasing the sensitivity of the assay format. Persons of ordinary skill in the art will appreciate that the design can be altered. For example, an affinity reagent specific for IgG3 antibodies can be immobilized to the solid surface. Any IgG3 antibodies are permitted to be bound and then the virus antigen can be contacted to the solid surface and allowed to bind to any antigen specific IgG3 antibody. The antigen can be detected directly or indirectly, as described above using detectably labeled antigen or secondary affinity reagent specific for the antigen, where the secondary affinity reagent (or a tertiary affinity reagent, etc.) is detectably labeled.

In another exemplary embodiment, the assay can be performed an absorbent strip that utilizes chromatographic flow to mix the sample analytes and reagents. For example, the strip can have a first region configured to receive the sample (e.g., in liquid form) wherein any antigen-specific IgG3 antibody is permitted to mix with detection reagents and flows along the strip past one or more readout regions with additional capture reagents that are immobilized thereto. Complexes of antigen-antigen-specific IgG3 antibody-affinity reagents will become immobilized in the one or more readout regions to present the detectable signal. In one particular embodiment, the IgG3 from the sample contacts free IgG3-specific affinity reagent and flows across a readout region comprising immobilized antigen, leaving any labeled antibody not specific for the antigen to continue flow past the read-out region. In alternative embodiment, the IgG3 from the sample contacts free antigen and flows across a readout region comprising immobilized IgG3-specific affinity reagent. The presence of captured IgG3 antibody can be detected directly (i.e., with detectably labeled detection reagent) or indirectly (i.e., with use of detectably labeled secondary or tertiary, etc., affinity reagent that ultimately bind to the detection reagent bound to IgG3 antibody), as described above.

In some embodiments, the method can be for binary detection, i.e., for the presence or absence of antigen-specific IgG3 antibodies. In other embodiments, depending on assay format, the method can comprise quantifying or estimating the amount or concentration of antigen-specific IgG3 antibodies. This can be helpful to assess a degree of neutralizing antibodies in the source of the sample, e.g., a subject. In some embodiments, the signal detected can be compared to a reference standard to ascertain the presence, absence, or amount of IgG3 antibodies and, thus the presence, absence, or amount of neutralizing antibodies in the sample. The reference standard can be readily established for any intended purpose using known amounts. The reference standard can be generated as part of the method or be pre-established.

As indicated above, in some embodiments the sample is a biological sample obtained from a subject. The method can further comprise obtaining the biological sample from the subject. The biological sample can be any sample that is likely to contain IgG3 antibodies from the particular subject. As indicated above, in humans the IgG antibodies, including the subclass of IgG3 antibodies, are predominantly released by plasma B cells into blood circulation. Thus, biological samples can be whole blood, plasma, serum. Additional biological samples useful in the methods and encompassed by the disclosure include sputum, nasopharyngeal swab specimen, buccal or oral swab specimen, rectal swab or stool specimen, spinal fluid, or processed derivatives thereof.

As described above, when the sample is a biological sample obtained from a subject, the determination of neutralizing antibodies in the biological sample via detection of IgG3 antibodies specific for the viral antigen, indicates that the subject has neutralizing antibodies against the virus. Thus, this method can be applied to broader methods of determining the infection and/or immunization status of the subject, which in turn can inform further treatment and vaccination strategies, or simply assess risk of infection based on current immunity to the virus. In an immediately relevant scenario, this is applicable to the COVID-19 pandemic.

Accordingly, in a further aspect the disclosure provides a method of determining whether a subject has neutralizing antibodies to a virus of the subfamily Orthocoronavirinae. The method comprises contacting a sample obtained from the subject with a virus antigen; and detecting binding of one or more IgG3 antibodies to the virus antigen. Detection of binding one or more IgG3 antibodies to the virus antigen indicates that the subject has neutralizing antibodies to the virus.

The method is applicable to viruses and viral antigens as described above in more detail. Briefly, in some embodiments, the virus is a coronavirus that causes the common cold, Middle East respiratory syndrome coronavirus (MERS-CoV), severe acute respiratory syndrome (SARS), or coronavirus disease 2019 (COVID-19). In a particular embodiment, the coronavirus is severe acute respiratory syndrome coronavirus 2 (SARS-CoV-2), as described above. When applied to a coronavirus, e.g., SARS-CoV-2, the viral antigen can be the spike protein, or an immunogenic fragment thereof. Exemplary fragments include the receptor binding domain of the S1 fragment.

Biological samples and formats for the assay are described above and are applicable to this aspect.

As indicated above, the assay can distinguish a binary status, i.e., the presence or absence of neutralizing antibodies, or can be quantify the level of neutralizing antibodies. The observed signal can be compared to a reference standard to facilitate quantification or, at the least, determine whether the level meets or exceeds a threshold level established for a particular purpose. For example, an assay can be performed on a biological sample obtained from a subject that is unvaccinated. The presence of, or a sufficient amount of, neutralizing antibodies can indicate a prior infection. In another embodiment, an assay can be performed on a biological sample obtained for a subject who previously received a vaccine. If the resulting signal meets or exceeds an established threshold, the subject is determined to have sufficient neutralizing antibodies with the current vaccination. If the established threshold is not met or exceeded, the subject is determined to be a candidate for additional vaccine booster. In some embodiments, the method would include administering a vaccine booster. Alternatively, the method can be useful to assess the efficacy of a particular vaccination regimen over time, e.g., by monitoring resultant neutralizing antibodies over various time points after inoculation. In another embodiment, the presence of neutralizing antibodies or lack thereof can be used to assess the risk of infection to the recipient from transplantation of SARS-CoV-2 positive organ donors. In yet further applications, the method can be used to assess or monitor a person's risk to severe disease in the midst of an active infection. Depending on the observed levels of neutralizing antibodies, a subject may be determined to be a candidate for more aggressive intervention (e.g., if the subject is unable to produce sufficient levels of neutralizing antibodies). Such interventions include administration of convalescent plasma, monoclonal antibodies, antiviral therapeutics (e.g., molnupiravir, paxlovid, fluvoxamine), therapeutics to reduce fever (e.g., acetaminophen, aspirin, ibuprofen, and the like), corticosteroids (e.g., dexamethasone, prednisone, methylprednisolone, and the like) and other therapeutics with an effect on viral infection (e.g., tocilizumab, remdesivir, baricitinib, anticoagulants, and the like.) Finally, additional non-pharmaceutical interventions, such as pre-emptive hospitalization and administration of fluids and rest, as well as preemptory precautions such as isolation, can be implemented or imposed.

In another aspect, the disclosure provides a kit useful for detecting the presence of neutralizing antibodies in a sample. The kit comprises at least an antigen of a virus of interest and an affinity reagent that specifically binds to IgG3 antibody. In some embodiments, one of the virus antigen and affinity reagent is immobilized on a solid substrate or surface. Thus, the kit can further comprise the solid substrate, such as an ELISA plate or strip device configured for lateral flow assays. The kit can also comprise buffers, blocking solutions, rinse solutions, and the like to facilitate the binding of antibodies in a sample to the antigen. Exemplary reagents are described in more detail below.

As indicated above, binding of the IgG3 to the antigen can be indirect, through the use of secondary or tertiary affinity reagents. The disclosure contemplates kits that encompasses such additional affinity reagents. The affinity reagent, whether primary, secondary, tertiary, etc., can be detectably labeled. Alternatively, the affinity reagent can be configured for labeling and the detectable label or moiety can be provided separately, or included within the kit but packaged separately. Detectable labels are described in more detail above.

Viruses, and virus antigens applicable to this aspect are described in more detail, which is not repeated here.

The kit can optionally include written indicia directing the performance the methods as described herein. Such instructions supplied in the kits of the invention are typically written instructions on a label or package insert (e.g., a paper sheet included in the kit), but machine-readable instructions (e.g., instructions carried on a magnetic or optical storage disk) are also acceptable.

The various reagents are packaged appropriately in containers to facilitate storage, shipping, handling, etc. Suitable packaging includes, but is not limited to, vials, bottles, jars, flexible packaging (e.g., sealed Mylar or plastic bags), and the like. A kit can have a sterile access port (e.g. the container can be an intravenous solution bag or a vial having a stopper pierceable by a hypodermic injection needle). Normally, the kit comprises a container and a label or package insert(s) on or associated with the container.

Additional definitions Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, N.Y. (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The term “affinity reagent” is an molecule or complex that specifically binds a target antigen of interest. The term “specifically binds” refers to, with respect to an antigen, the preferential association of an affinity reagent, in whole or part, with a specific antigen, such as a viral antigen or peptide sequence characteristic of an antibody subclass (e.g., IgG3). A specific binding affinity reagent binds substantially only to a defined target, such as a specific chromatin associated factor or marker. It is recognized that a minor degree of non-specific interaction may occur between a molecule, such as a specific affinity reagent, and a non-target antigen. Nevertheless, specific binding can be distinguished as mediated through specific recognition of the antigen. Specific binding typically results in greater than 2-fold, such as greater than 5-fold, greater than 10-fold, or greater than 100-fold increase in amount of bound affinity reagent (per unit time) to a target antigen, such as compared to a non-target antigen. A variety of immunoassay formats are appropriate for selecting affinity reagent specifically reactive with a particular antigen. For example, solid-phase ELISA immunoassays are routinely used to select antibodies specifically immunoreactive with a protein. See Harlow & Lane, Antibodies, A Laboratory Manual, Cold Spring Harbor Publications, New York (1988), for a description of immunoassay formats and conditions that can be used to determine specific reactivity.

For example, the affinity reagent can be an antibody or an antibody-like molecule.

As indicated above, an “antibody” is a polypeptide ligand that includes at least a light chain or heavy chain immunoglobulin variable region and specifically binds an epitope of an antigen, such as a viral antigen or other molecules (e.g., another antibody). In the context of affinity reagents, as used herein, the term “antibody” encompasses antibodies, derived from any antibody-producing mammal (e.g., mouse, rat, rabbit, and primate including human), that specifically bind to an antigen of interest (e.g., a chromatin associated marker or another affinity reagent). Exemplary antibody types include multi-specific antibodies (e.g., bispecific antibodies), humanized antibodies, murine antibodies, chimeric, mouse-human, mouse-primate, primate-human monoclonal antibodies, and anti-idiotype antibodies.

Canonical antibodies can be composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. In the context of an affinity reagent, the term “antibody-like molecule” includes functional fragments of intact antibody molecules, molecules that comprise portions of an antibody, or modified antibody molecules, or derivatives of antibody molecules. Typically, antibody-like molecules retain specific binding functionality, such as by retention of, e.g., with a functional antigen-binding domain of an intact antibody molecule. Preferably antibody fragments include the complementarity-determining regions (CDRs), antigen binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above antibody-based affinity reagent does not have to be naturally occurring or naturally derived, but can be further modified to, e.g., reduce the size of the domain or modify affinity for the antigen as necessary. For example, complementarity determining regions (CDRs) can be derived from one source organism and combined with other components of another, such as human, to produce a chimeric molecule that avoids stimulating immune responses in a subject.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Once a monoclonal antibody is identified for inclusion within the bi-specific molecule, the encoding gene for the relevant binding domains can be cloned into an expression vector that also comprises nucleic acids encoding the remaining structure(s) of the bi-specific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the C_{H}1 domain of the heavy chain. Further, the antibodies of the present invention can also be generated using various phage display methods known in the art.

The affinity reagent can also be an aptamer. As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phosphodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/target binding.

Peptide aptamers are protein molecules that often contain a peptide loop attached at both ends to a protamersein scaffold. The loop typically has between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

Designed ankyrin repeat proteins (DARPins) are engineered antibody mimetic proteins that can have highly specific and high affinity target antigen binding and, thus, can also serve as affinity reagents as disclosed herein. DARPins are typically based on natural ankyrin repeat proteins and comprise at least three repeat motifs. Repetitive structural units (motifs) form a stable protein domain with a large potential target interaction surface. Typically, DARPins comprise four or five repeats, of which the first (N-capping repeat) and last (C-capping repeat) serve to shield the hydrophobic protein core from the aqueous environment. DARPins often correspond to the average size of natural ankyrin repeat protein domains. DARPins can be screened and engineered starting from encoding libraries of randomized variations. Once desired antigen binding characteristics are discovered, the encoding DNA can be obtained. Library screening and use can incorporate ribosome display or phage display.

The terms “subject,” “individual,” and “patient” are used interchangeably herein to refer to a subject, such as a mammal, being assessed for treatment and/or being treated. In certain embodiments, the mammal is a human. The terms “subject,” “individual,” and “patient” encompass, without limitation, individuals having a viral inventions, such as coronavirus infection (e.g., COVID-19). While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., COVID-19), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, to improve clinical outcomes, to decrease occurrence of symptoms, to improve quality of life, to lengthen disease-free status, to stabilize, to prolong survival, to arrest or inhibit development of the symptoms or conditions associated with a disease or condition (e.g., COVID-19), or any combination thereof. The term “therapeutic effect” refers to the reduction, elimination, or prevention of the disease or condition, symptoms of the disease or condition, or side effects of the disease or condition in the subject.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill in the art will recognize that individual substitutions, deletions or additions to a peptide, polypeptide, or protein sequence which alters, adds or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:

(1) Alanine (A), Serine (S), Threonine (T),

(2) Aspartic acid (D), Glutamic acid (E),

(3) Asparagine (N), Glutamine (Q),

(4) Arginine (R), Lysine (K),

(5) Isoleucine (I), Leucine (L), Methionine (M), Valine (V), and

(6) Phenylalanine (F), Tyrosine (Y), Tryptophan (W).

As used herein, the term “nucleic acid” refers to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxymethylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are provided to illustrate certain particular features and/or embodiments of the disclosure. The examples should not be construed to limit the disclosure to the particular features or embodiments described.

### Example 1

This Example describes compare combinations of four viral antigens and five human antibody isotype ELISA platforms in control and unknown exposure populations coupled to classic viral neutralization studies, revealing that a specific combination approach of antibody isotype and neutralization activity of virus-specific antibody best determines exposure and possible protection to SARS-CoV-2.

### Results

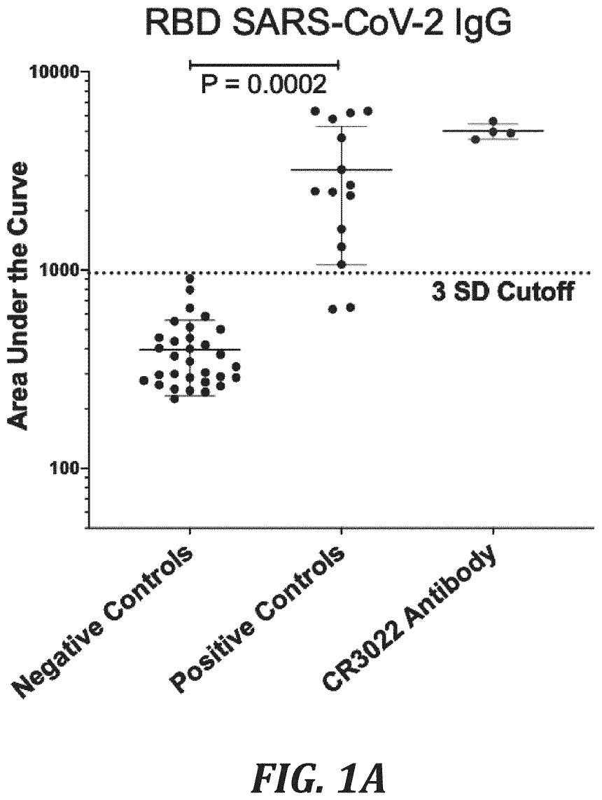

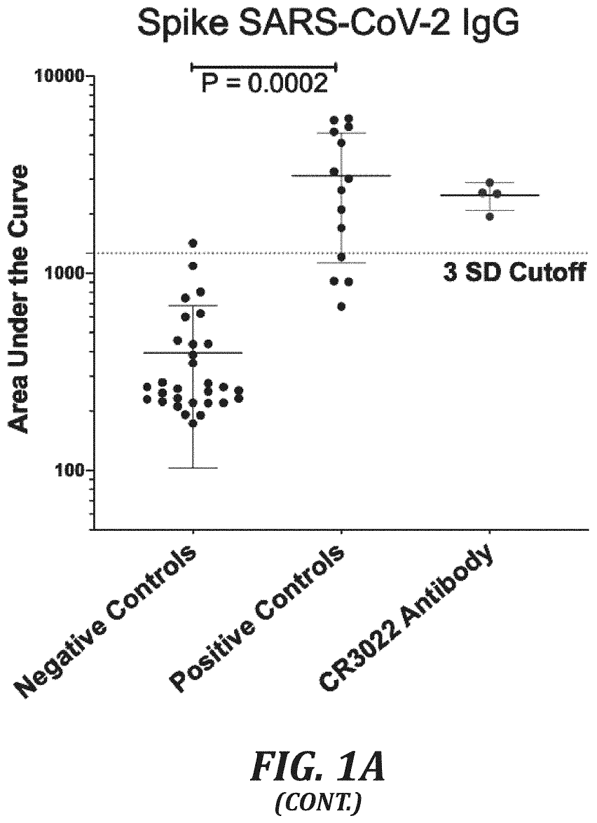

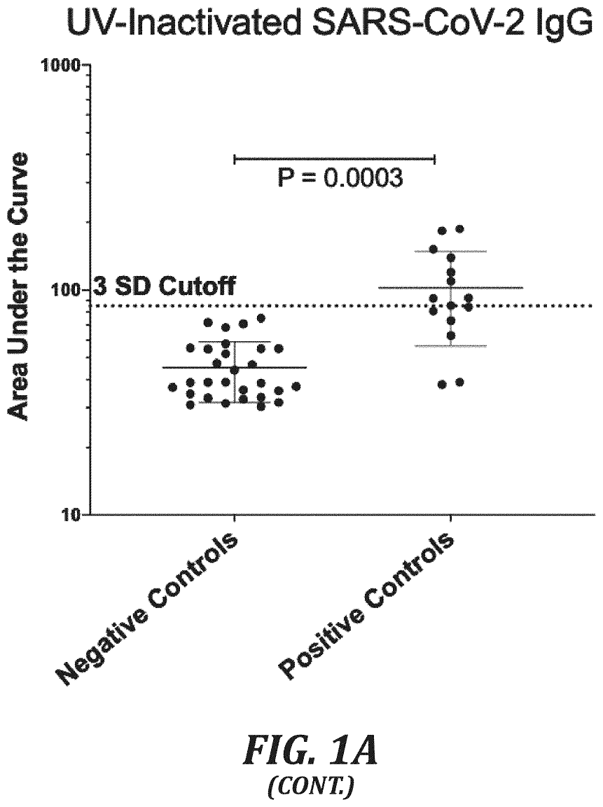

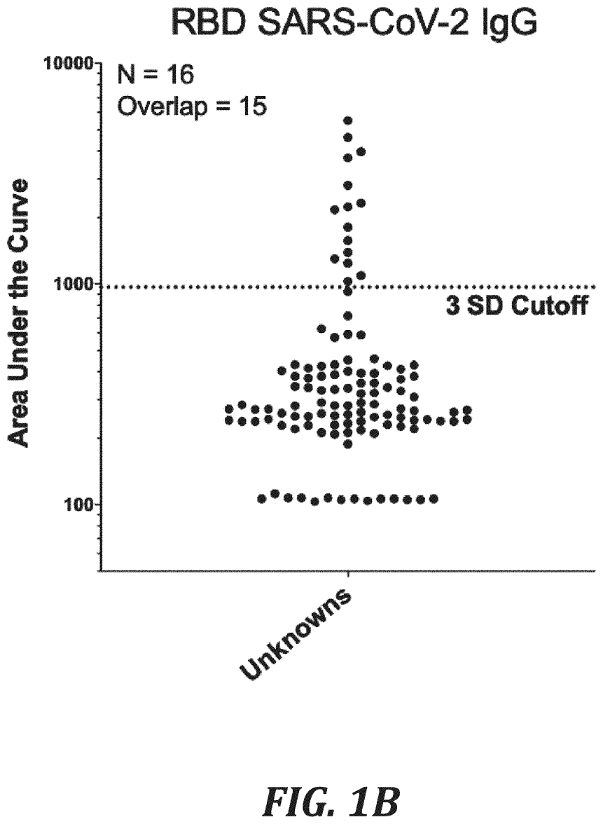

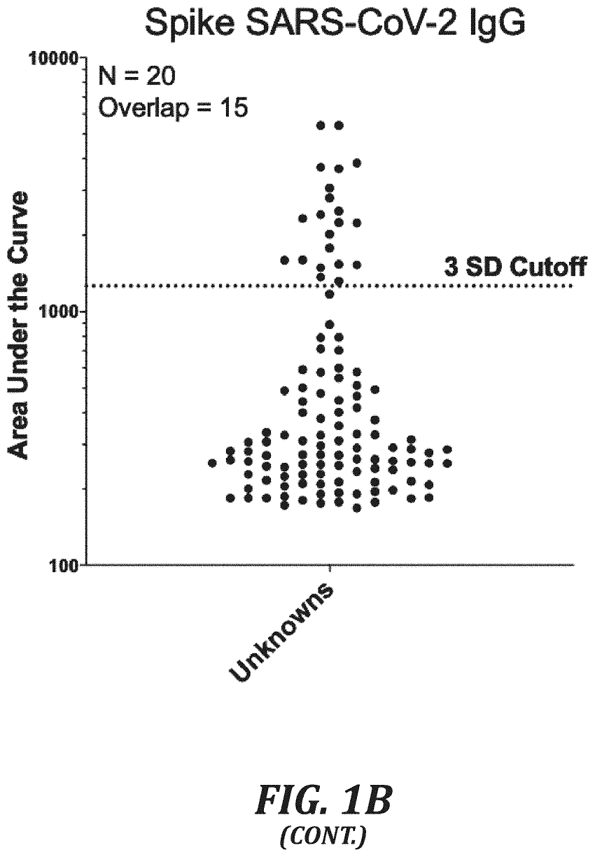

To evaluate the presence of SARS-CoV-2 specific antibodies and neutralization capability, subjects were enrolled from the greater Seattle area for plasma collection and antibody testing. Positive controls included 15 symptomatic RT-PCR SARS-CoV-2 positive individuals enrolled around 21 days after the positive PCR test result. All of the positive controls had mild symptoms and none were hospitalized (TABLE 1). Thirty negative controls were obtained from a blood bank as deidentified plasma collected prior to Dec. 1, 2019. Unknown samples consisted of two cohorts: Group A and Group B. Group A included 14 subjects with known exposure to confirmed SARS-CoV-2 infected, symptomatic individuals. Group B subjects were randomly recruited from the community with unknown exposure to or infection with SARS-CoV-2. Serum samples were collected from Mar. 26 to Apr. 15, 2020. Positive and negative control plasma samples were assessed by ELISAs based on three SARS-CoV-2 antigens: RBD, spike (Walls A C, et al. Structure, Function, and Antigenicity of the SARS-CoV-2 Spike Glycoprotein. Cell 2020; 181:281-92.e6), and full SARS-CoV-2 UV-inactivated viral particles (FIG. 1A). Serial dilutions of each sample generated AUC values plotted with associated subject cohorts. Antibody-positive samples were designated as any samples with an AUC above the mean+3SDs of the AUCs of the negative control samples (Stadlbauer D, et al. SARS-CoV-2 Seroconversion in Humans: A Detailed Protocol for a Serological Assay, Antigen Production, and Test Setup. Curr Protoc Microbiol 2020; 57:e100). RBD and spike viral antigen of total IgG ELISAs demonstrated the clearest separation between positive and negative controls. Given the comparatively improved identification of control samples by RBD and spike ELISAs, no further testing of the UV-inactivated whole SARS-CoV-2 virus platform were performed. Calculations for sensitivity, specificity, positive predictive value (PPV), negative predictive value (NPV), and associated 95% confidence intervals (CI) were determined based on the negative and positive control samples (TABLE 2). RBD IgG outperformed spike IgG in all measured parameters including specificity, sensitivity, PPV, and NPV. Unknowns assessed for IgG reactivity against RBD and spike identified 16 and 20 positive samples, respectively. Fifteen of the positive samples were identified by both platforms.

TABLE 1
Positive control subjects information. Shown are the
ages, sex, and symptom profiles of our positive control
samples. All samples were collected >21 days after positive
nasal swab PCR test for SARS-CoV-2. No positive subjects
were hospitalized for their symptoms but they did all
experience symptoms. No = 0; yes = 1. Female = 2; male = 3.

| | | | | | | | Diffi- | | |
|---|---|---|---|---|---|---|---|---|---|
| | | | | Run- | | | culty | | |
| Sub- | | | | ny | Fa- | Muscle | Breath- | | |
| ject | Fever | Chills | Cough | Nose | tigue | Aches | ing | Age | Sex |
| 1 | 1 | 1 | 1 | 1 | 1 | 0 | 1 | 67 | 3 |
| 2 | 0 | 0 | 1 | 1 | 1 | 0 | 0 | 47 | 2 |
| 3 | 1 | 1 | 1 | 1 | 1 | 1 | 1 | 43 | 3 |
| 4 | 1 | 1 | 1 | 1 | 1 | 1 | 0 | 46 | 2 |
| 5 | 1 | 1 | 1 | 1 | 1 | 1 | 1 | 42 | 2 |
| 6 | 1 | 1 | 0 | 1 | 1 | 1 | 0 | 37 | 2 |
| 7 | 0 | 0 | 0 | 1 | 1 | 1 | 1 | 28 | 2 |
| 8 | 0 | 0 | 1 | 1 | 0 | 0 | 0 | 71 | 3 |
| 9 | 1 | 1 | 1 | 1 | 1 | 1 | 1 | 67 | 2 |
| 10 | 0 | 1 | 1 | 1 | 1 | 1 | 0 | 34 | 2 |
| 11 | 0 | 0 | 0 | 1 | 1 | 0 | 0 | 51 | 3 |
| 12 | 1 | 1 | 0 | 0 | 1 | 1 | 0 | 53 | 2 |
| 13 | 0 | 0 | 0 | 0 | 1 | 0 | 0 | 47 | 2 |
| 14 | 1 | 1 | 1 | 1 | 1 | 1 | 1 | 43 | 2 |
| 15 | 1 | 0 | 0 | 0 | 1 | 0 | 0 | 57 | 2 |

TABLE 2
Summary of ELISA platform performance using positive and negative control samples.
Sensitivity, specificity, positive predictive value (PPV), negative predictive value (NPV),
and associated 95% confidence intervals (CI) calculated based on positive and negative
controls. 95% CI range is shown in parenthesis following the calculated values.

| | | | Positive | Negative |
|---|---|---|---|---|
| | | | Predictive | Predictive Value |
| | Specificity | Sensitivity | Value (PPV) | (NPV) |
| RBD | 87% (62-98%) | 100% (89-100%) | 100% (77-100%) | 94% (80-99%) |
| IgG | | | | |
| RBD | 73% (48-89%) | 97% (83-100%) | 92% (65-100%) | 88% (73-95%) |
| IgM | | | | |
| Spike | 67% (42-85%) | 97% (83-100%) | 91% (62-100%) | 85% (70-94%) |
| IgG | | | | |
| Spike | 60% (36-80%) | 97% (83-100%) | 90% (60-100%) | 83% (67-92%) |
| IgM | | | | |
| Spike | 71% (45-88%) | 100% (87-100%) | 100% (72-100%) | 88% (73-95%) |
| IgG1 | | | | |
| Spike | 93% (70-100%) | 97% (83-100%) | 93% (70-100%) | 97% (83-100%) |
| IgG3 | | | | |
| Spike | 67% (42-85%) | 100% (89-100%) | 100% (72-100%) | 86% (71-94%) |
| IgA | | | | |
| NP IgG | 93% (70-100%) | 97% (83-100%) | 93% (70-100%) | 97% (83-100%) |

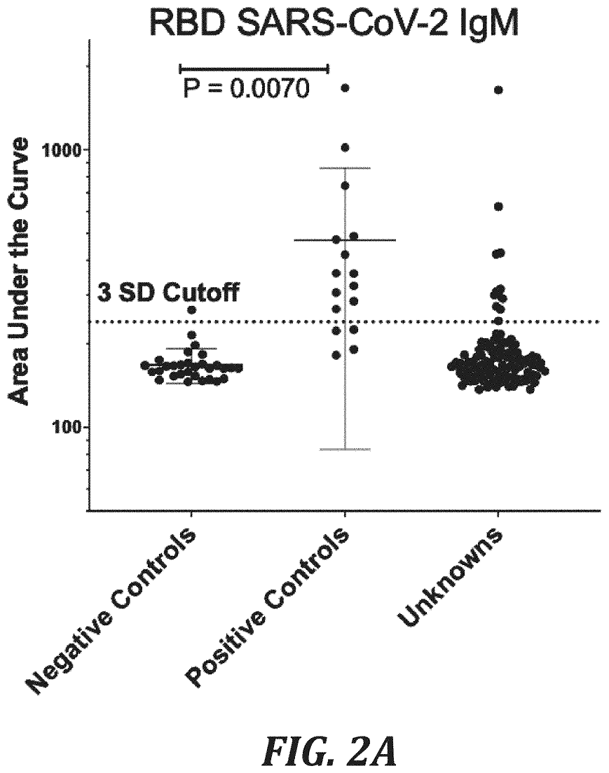

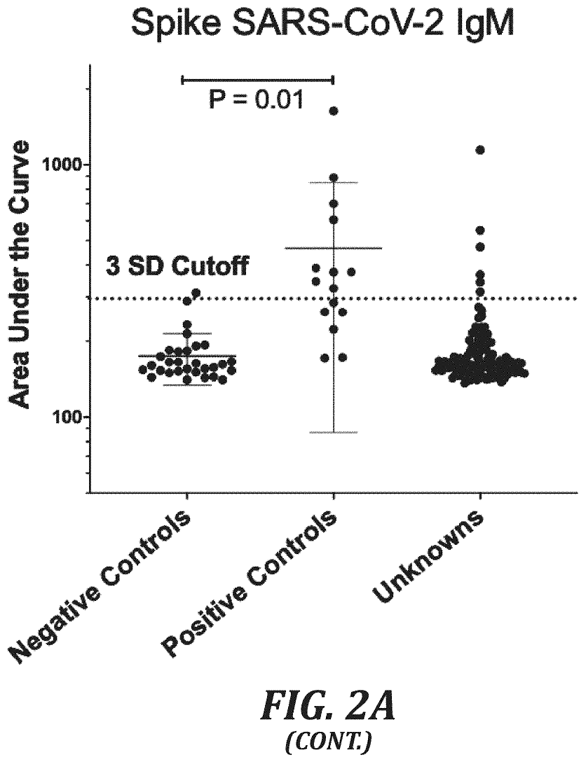

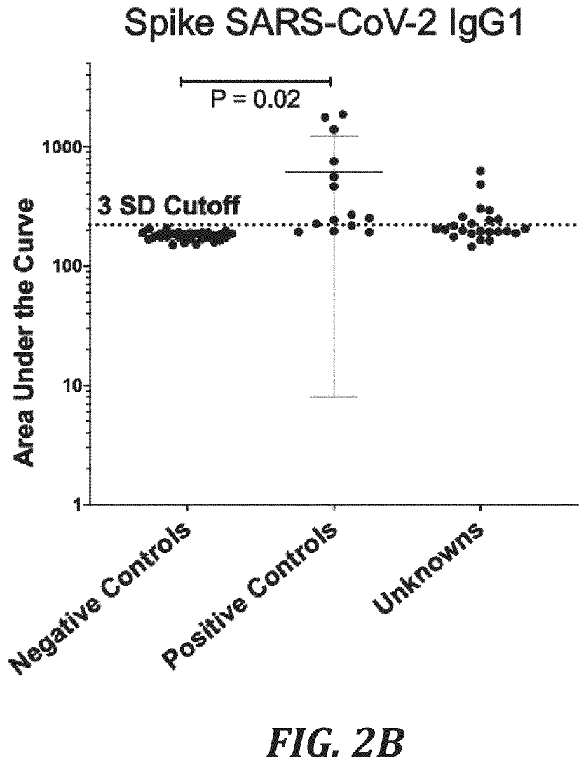

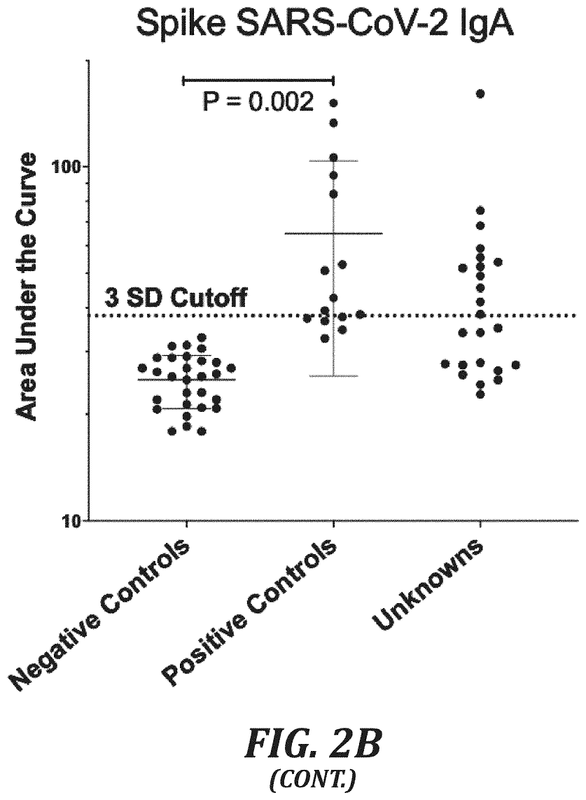

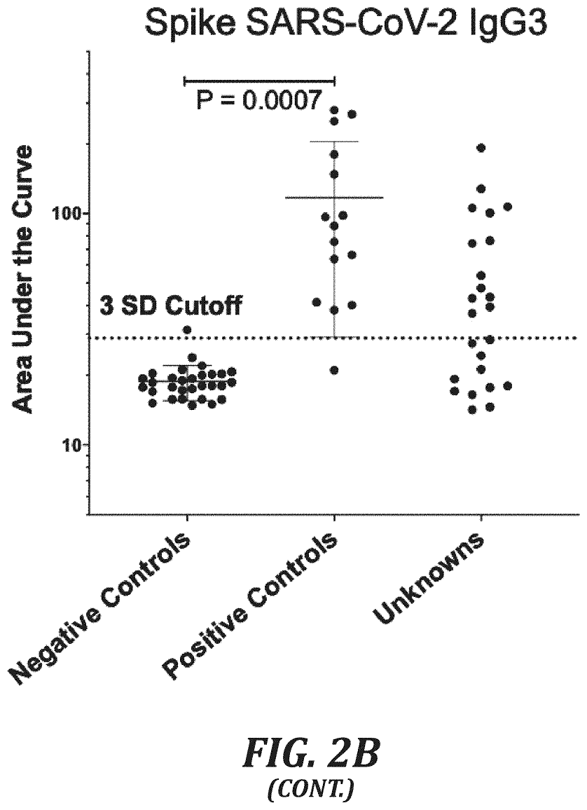

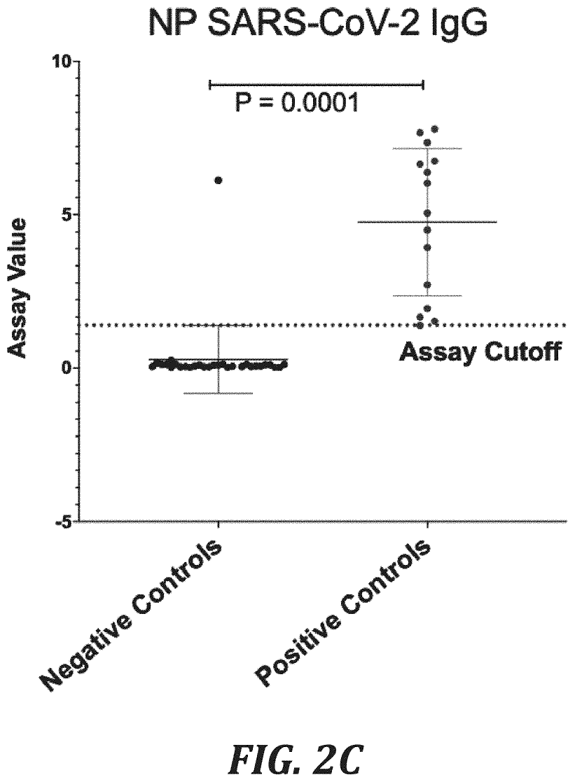

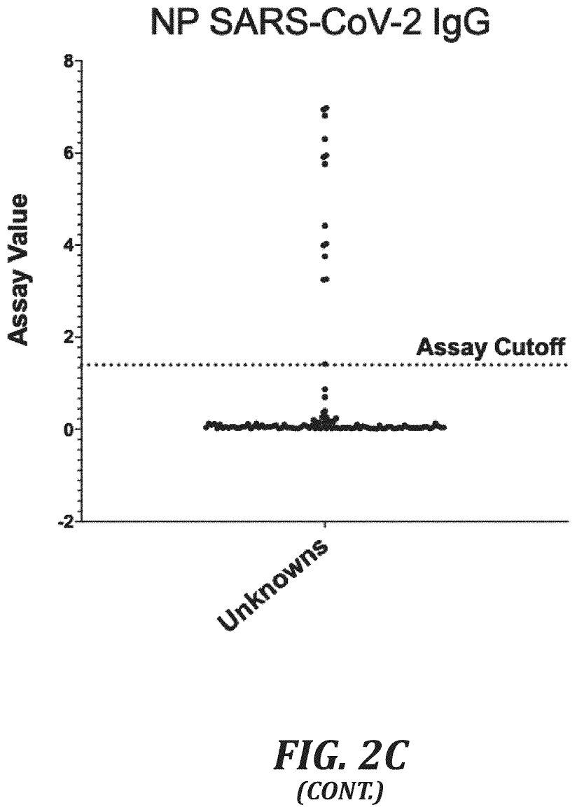

Next, test performance of a range of antibody isotypes against SARS-CoV-2 was assessed. For the positive and negative control samples, IgM reactivity was decreased compared to IgG for both RBD and spike (FIG. 2A). Comparison of assay performance measures between IgG and IgM assays for RBD and spike yielded decreased sensitivity, specificity, PPV, and NPV for the IgM assays (TABLE 2). Analyses of the unknowns (Group A and B) with the IgM assays identified fewer positive samples compared to their IgG counterpart. However, all of the positives that were identified on the IgM assays were also identified on the IgG RBD and spike assays with one exception. All positive and negative controls as well as any unknowns identified as positive for IgG antibody titers against RBD or spike were additionally assessed for IgG1, IgG3, and IgA spike specific antibodies (FIG. 2B). IgG3 demonstrated the highest sensitivity and NPV while IgG1 and IgA had relatively superior specificity and PPV. The spike IgG3 platform detected positive reactivity in 15 of the 20 unknown samples identified as positive by the IgG RBD and spike platforms.

The samples were tested using an FDA emergency use-authorized ELISA based on SARS-CoV-2 NP platform to identify NP-specific IgG responses. The NP IgG platform resulted in similar separation of positive and negative control subjects compared to RBD IgG, spike IgG, and spike IgG3 (FIG. 2C). The NP IgG platform demonstrated similar specificity, sensitivity, PPV, and NPV compared to spike IgG3 and improved sensitivity compared to both RBD and spike IgG (TABLE 2). The NP IgG platform identified 14 unknowns as antibody-positive all of which were also identified by the RBD IgG, spike IgG, and spike IgG3 platforms.

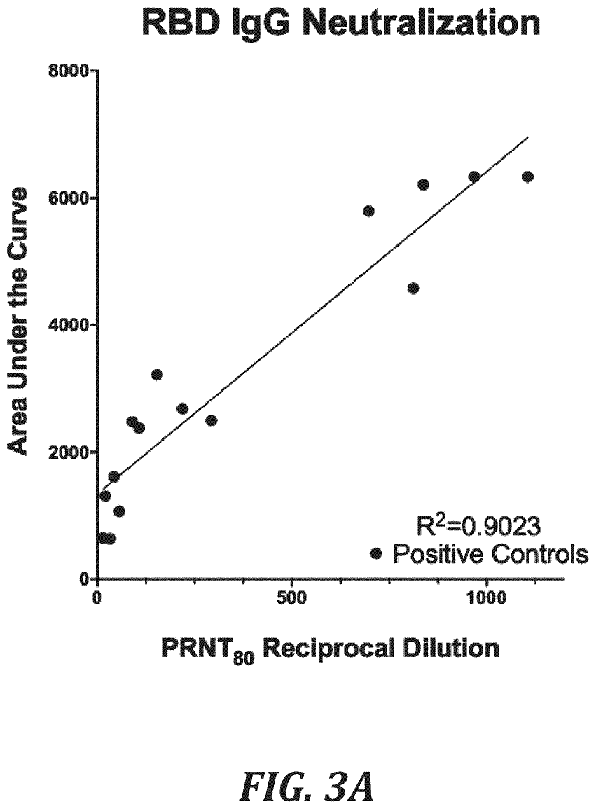

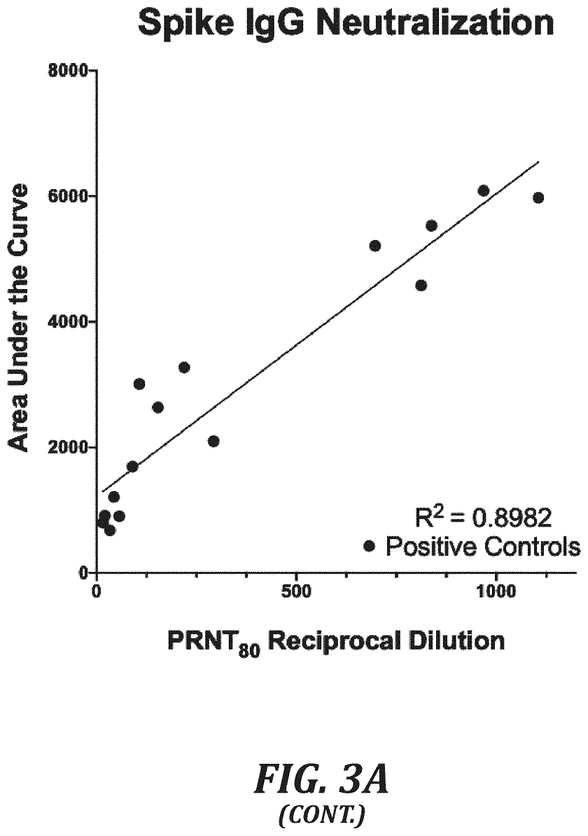

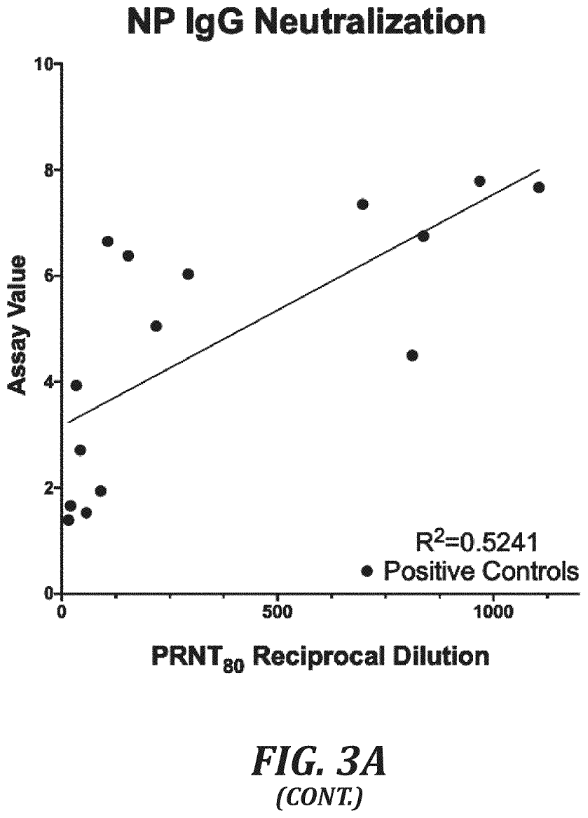

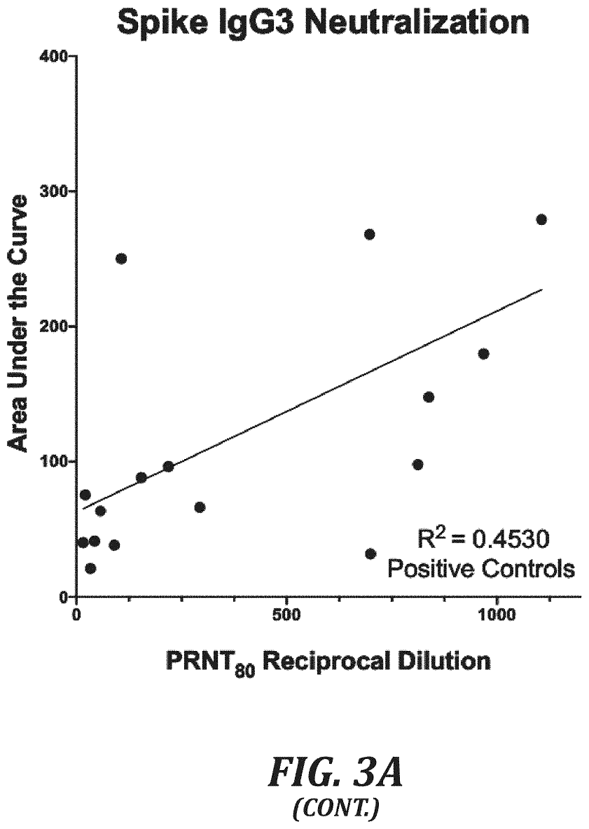

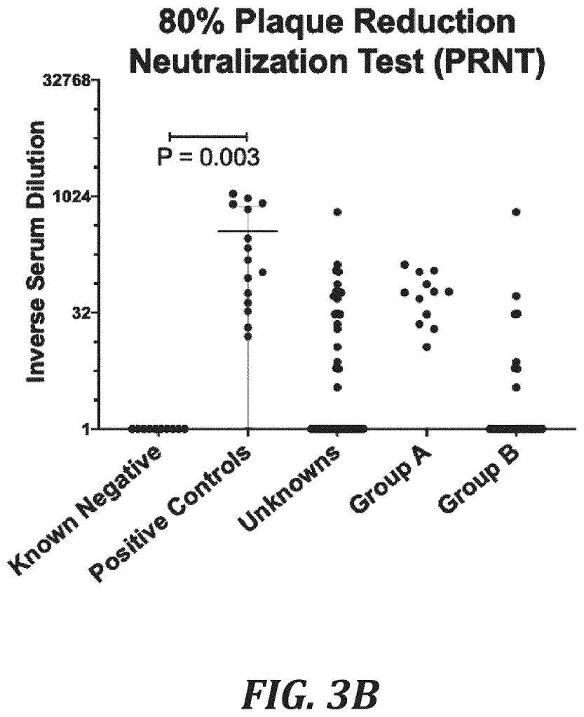

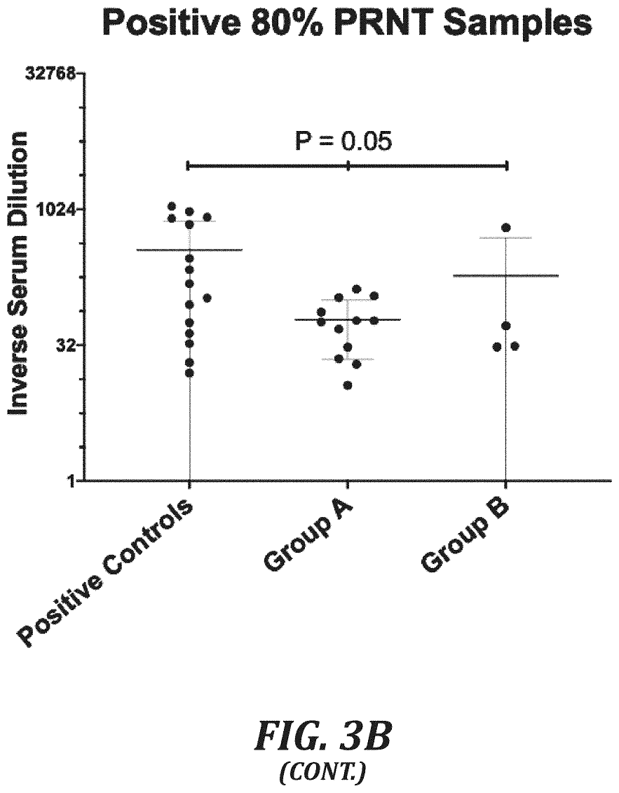

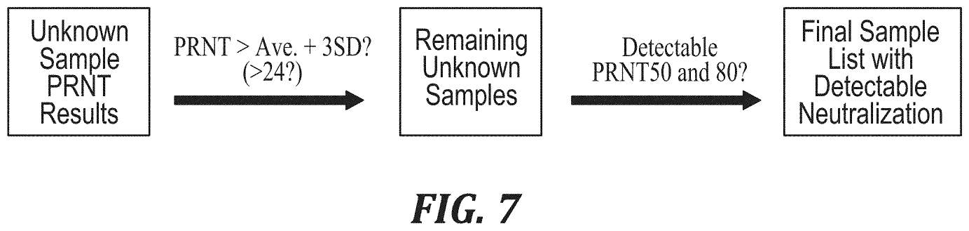

PRNT analysis against SARS-CoV2/WA-1 isolate was used to assess antibody function via virus neutralization activity (FIGS. 3A and 3B). PRNT was performed on the following samples: all positive controls, 10 negative control samples (5 with ‘antibody-positive’ cut-off designations), any RBD/spike/NP IgG identified ‘positive’ unknowns, and 10 unknown samples near the cut-off but technically ‘negative’. For positive control subjects, the correlation between PRNT 80% (PRNT₈₀) reciprocal dilutions and ELISA AUC quantitative values (FIG. 3A) was assessed. RBD-specific IgG ELISA results showed the strongest correlation between the magnitude of ELISA antibody signal to the strength of neutralization followed by >spike IgG>NP>spike IgG3. A cutoff was established for detectable neutralization for unknown samples based on positive and negative control PRNT results (TABLE 3, FIG. 7, and TABLE 4). Detectable neutralization sample cut offs require i) end point dilution levels 3SD greater than the mean value for negative control samples, and ii) neutralization titers present at both PRNT 50% (PRNT₅₀) and PRNT₈₀. FIG. 3B demonstrates PRNT₈₀ values in the left panel, with samples meeting the neutralization criteria in the right panel. 12 of the 14 Group A subjects, and 4 of the Group B subjects showed detectable neutralization. The 10 unknown samples near the ELISA cut-offs but designated as ‘negative’ were also negative for virus neutralization.

TABLE 3
Plaque neutralization reduction test (PRNT) cut off criteria.
Shown are the PRNT negative and positive controls.
The flow chart in FIGURE 7 demonstrates the
values of the resultant cut off criteria.

| Negative | | | Positive | | |
|---|---|---|---|---|---|
| Controls | PRNT 50 | PRNT 80 | Controls | PRNT 50 | PRNT 80 |
| 1 | 1.00 | 1.00 | 1 | 1652.76 | 811.95 |
| 2 | 1.00 | 1.00 | 2 | 1200.50 | 292.69 |
| 3 | 1.00 | 1.00 | 3 | 56.08 | 33.38 |
| 4 | 1.00 | 1.00 | 4 | 169.85 | 106.85 |
| 5 | 1.30 | 1.00 | 5 | 40.00 | 20.56 |
| 6 | 22.02 | 1.00 | 6 | 156.07 | 56.98 |
| 7 | 1.00 | 1.00 | 7 | 551.79 | 219.09 |
| 8 | 1.00 | 1.00 | 8 | 47.55 | 15.74 |
| 9 | 1.00 | 1.00 | 9 | 4536.78 | 968.29 |
| 10 | 1.00 | 1.00 | 10 | 443.02 | 90.01 |
| | | | 11 | 1581.40 | 837.65 |
| Average | 3 | | 12 | 2075.02 | 1105.56 |
| Standard | 7 | | 13 | 437 | 43 |
| Deviation | | | | | |
| Ave + 3D | 24 | | 14 | 584 | 154 |
| | | | 15 | 2957 | 697 |

TABLE 4
Unknown samples with detectable neutralization.
Shown are the unknown samples meeting cut off
criteria for detectable neutralization.

| Sample | PRNT50 | PRNT80 |
|---|---|---|
| 1 | 116.20 | 30.70 |
| 2 | 37.88 | 31.23 |
| 3 | 131.93 | 52.59 |
| 4 | 2560.45 | 642.48 |
| 5 | 57.11 | 30.48 |
| 6 | 130.74 | 74.64 |
| 7 | 474.49 | 133.95 |
| 8 | 34.84 | 11.51 |
| 9 | 173.47 | 59.83 |
| 10 | 163.56 | 108.19 |
| 11 | 40.89 | 22.71 |
| 12 | 90.78 | 48.33 |
| 13 | 100.26 | 59.81 |
| 14 | 46.43 | 19.70 |
| 15 | 211.31 | 112.38 |
| 16 | 102.90 | 58.01 |

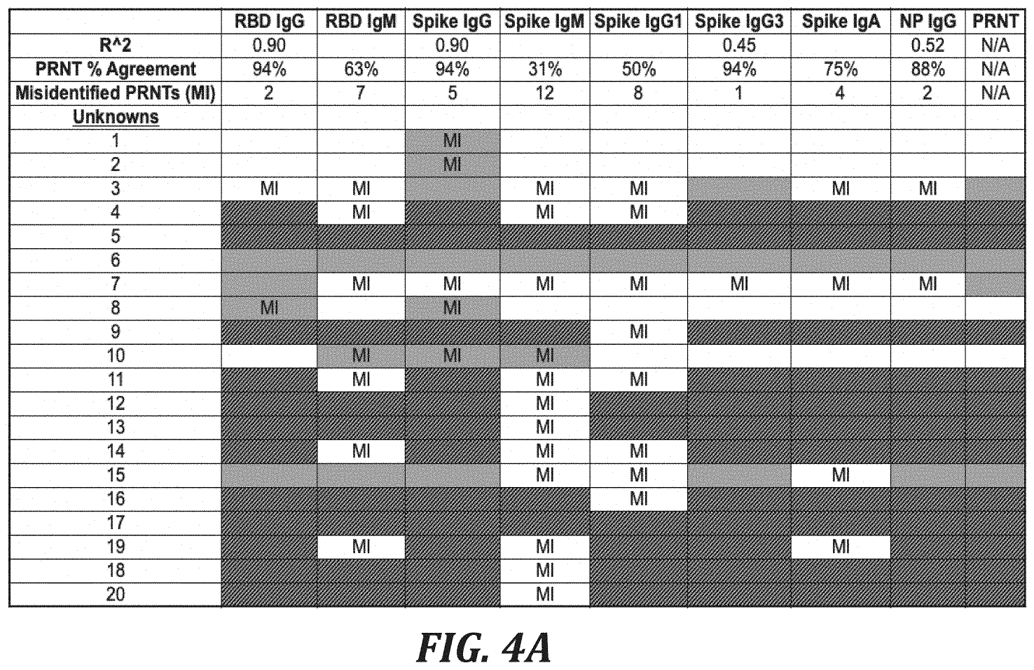

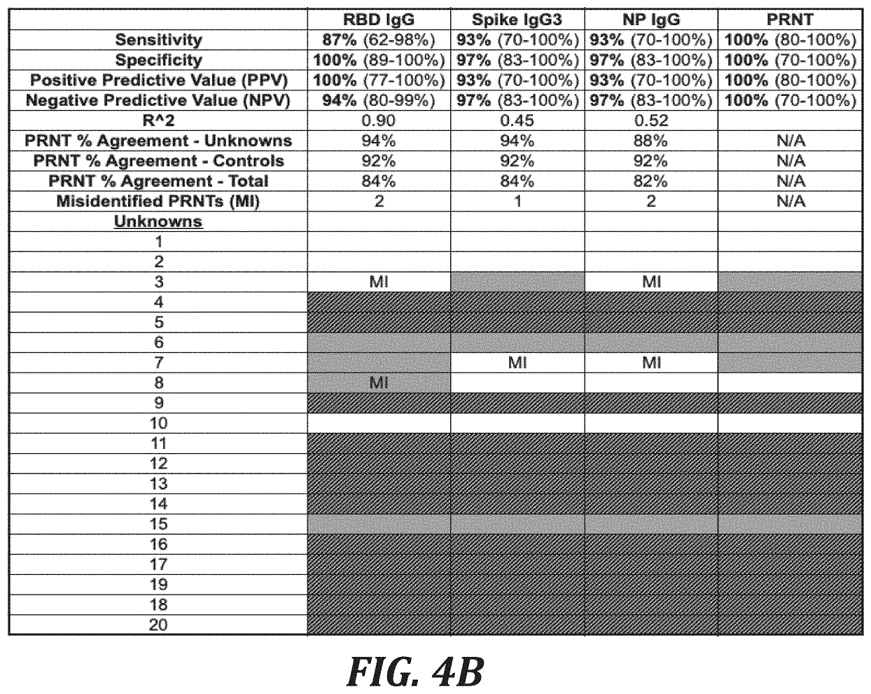

SARS-CoV-2 PRNT results provide key context to interpret ELISA results and functional evidence of viral neutralization. TABLE 2 illustrates the sensitivity, specificity, PPV, and NPV measurements based on the positive and negative control subjects' antibody reactivity to each of the ELISA platform assays. Sensitivity is superior for spike IgG3 and NP IgG; therefore, these two platforms will identify more true positive samples compared to RBD and spike IgG. Specificity is best for RBD IgG, spike IgG1, and spike IgA platforms resulting in better identification of true negatives. Overall, three platforms have the most desirable testing characteristics including RBD IgG, spike IgG3, and NP IgG. FIGS. 4A and 4B are tables that summarize and connect the ELISA and PRNT data. The R² correlation between the magnitude of specific ELISA platform antibody detection and neutralization strength is highest for RBD and spike IgG. PRNT percent agreement was calculated for each platform as the number of subjects positive by specific ELISA platforms that were also positive for neutralization for controls samples and unknowns; RBD IgG and spike IgG3 had the highest percent agreement with PRNTs across all cohorts. Subject misidentifications are discordant results between specific ELISA platforms and neutralization not accounted for by the PRNT percent agreement calculations. The spike IgG3 platform had the least number of misidentifications followed by NP IgG and RBD IgG. FIG. 4B compares the top three performing assays across all measures of performance, including RBD IgG, spike IgG3, and NP IgG. Overall, the spike IgG3 assay demonstrated the highest accuracy for identifying serologically positive individuals with detectable neutralizing antibody activity; NP IgG and RBD IgG platforms were slightly inferior in their ability to predict neutralization in our sample set.

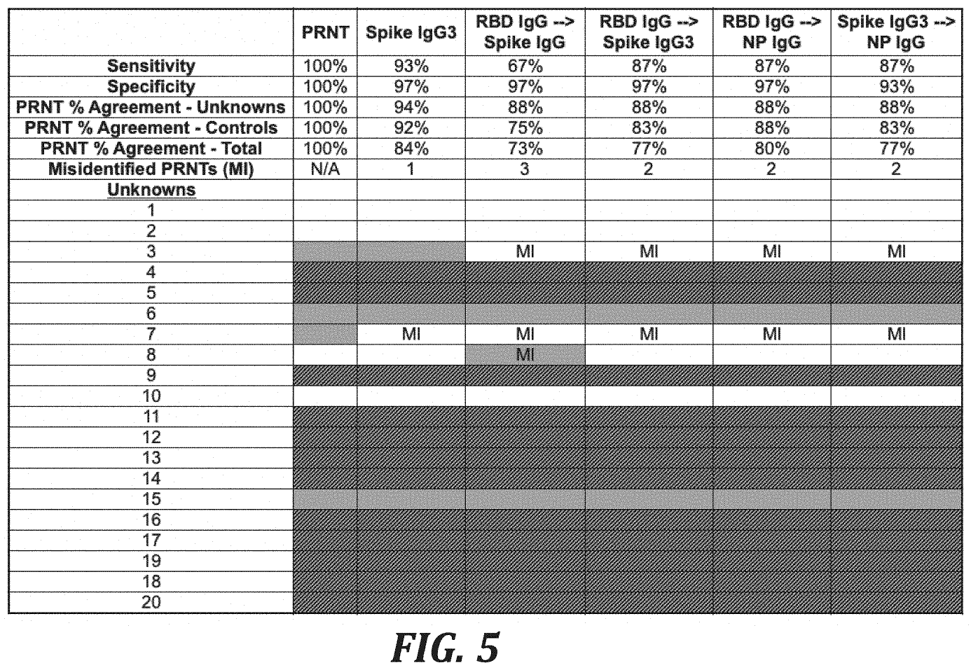

Sequential ELISA assay testing platforms have been proposed to increase sensitivity and specificity of SARS-CoV-2 antibody testing. FIG. 5 compares sequential ELISA assays using sensitivity, specificity, PRNT agreement and PRNT misidentifications. No two sequential ELISA assays outperformed spike IgG3.

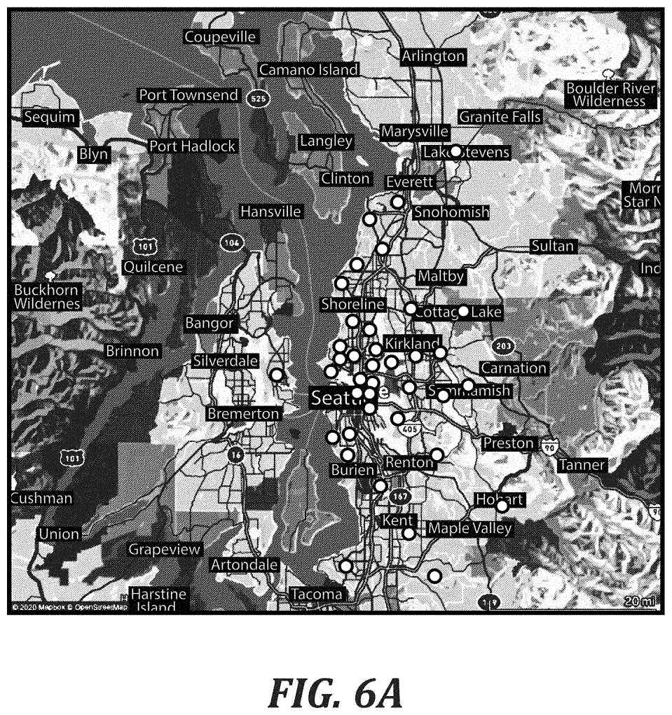

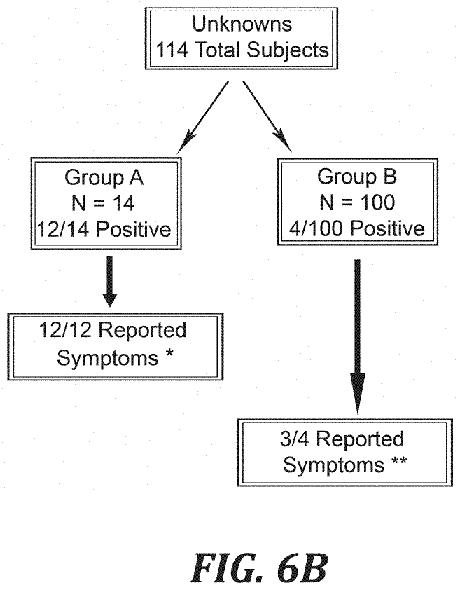

Using detectable neutralization to identify positive unknown samples, the prevalence of individuals in the Seattle area with neutralizing antibodies against SARS-CoV-2 we estimated as of March-April 2020 (FIGS. 6A and 6B). FIG. 6A is a map of the greater Seattle area with subjects designated by zip code. FIG. 6B shows the number of positives identified from group A and B as well as those that reported symptoms. Prevalence was estimated using a weighted logistic regression model adjusting for age and sex (see figure legend). The prevalence of individuals with SARS-CoV-2 antibodies at the time of this study was estimated at 3.5% with a 95% CI of 1.3-7.3%. Comparison of exposed and unexposed cohorts shows a significant increase in the frequency of detection of neutralizing SARS-CoV-2 antibodies in those with a known exposure to infected individuals.

### DISCUSSION

This study shows that a two-tiered testing strategy of ELISA followed by PRNT of positive ELISA samples is the most accurate way to assess humoral immunity to SARS-CoV-2, and that anti-spike IgG3 is the best predictor of presence of neutralizing antibodies. Comprehensive analyses of multiple ELISA SARS-CoV-2 platforms coupled with the gold standard of viral neutralization testing were completed to determine a testing strategy most likely to identify true positives and best predictive of SARS-CoV-2 neutralization. Relying on results from a single analysis of testing performance or testing only in control populations did not provide enough information to assess test performance. It was found that having paired neutralization studies in control and unknown populations was key to interpreting results of the ELISAs for SARS-CoV-2.

Three ELISA platforms are top performers when high sensitivity and specificity, high PRNT agreement, and low misidentification of subjects were prioritized: RBD IgG, spike IgG3, and NP IgG. Spike IgG3 surpasses RBD IgG because of its balance of high sensitivity and specificity as well as its superior prediction of neutralization in our unknown population (MI=1/20 vs. 2/20). Of note, the R² for RBD IgG was higher compared to the lower correlation for spike IgG3. However, this difference is challenging to interpret given the finding in other studies with variable and waning correlation with RBD IgG to neutralization (R² ranging from 0.5-0.8) when assessed in larger positive control groups. The positive controls in this study were collected around the same time from infection onset and may explain the high correlation. In assessing unknown populations with the present platforms, it was found that despite the higher correlation with PRNT, RBD IgG performed less well at predicting neutralization compared to spike IgG3. With the variable reported values of R² for RBD and spike platforms, this outcome measure does not appear to be a dependable measurement of performance for SARS-CoV-2 ELISA platforms to predict neutralization.

Across the ELISA platforms investigated, the data supports the use of spike IgG3 as the best initial screening test to predict neutralization. Many of the parameters of test performance were similar for RBD IgG, spike IgG3, and NP IgG. However, spike IgG3 edged out both NP and RBD IgG with the ability to predict neutralization (% PRNT agreement and misidentification). RBD and NP IgG platforms already have FDA emergency approval, with the NP IgG platform having the highest combined sensitivity and specificity as well as the most comprehensive validation. The NP IgG assay is likely to be limited in its ability to predict antibody neutralization activity due to internal NP localization within the intact virion being inaccessible to antibodies in vivo. This consideration is especially relevant for vaccine responses, as current vaccines being launched for human application are designed to generate immunity against the virion spike protein. Spike IgG3 platforms have not been developed but represent a highly promising platform given that IgG3 isotypes are thought to be more effective at viral neutralization compared to other IgG subtypes.

Classic PRNTs are expensive and time-consuming due to laboratory biosafety requirements. However, development of pseudovirus systems have demonstrated high correlation with the SARS-CoV-2 PRNT assay, which would allow for high-throughput sample analysis of neutralizing antibodies. Coupling of a pseudovirus system with spike IgG3 ELISA would provide an accurate and practical two-tiered testing method to use within a standard clinical laboratory to assess for possible correlates of immunity against SARS-CoV-2.

Lastly, the prevalence of individuals with detectable SARS-CoV-2 neutralizing antibodies in the Seattle area was estimated. The United States Center for Disease Control (CDC) reported a prevalence of 1.13% in samples obtained from the same time frame but their study suffers from sampling bias given the unknown population utilized. The CDC study prevalence estimate could be either an over or underestimate. It is believed that the 95% CI (1.3-7.3%) range found in this study offers a more accurate estimate of SARS-CoV-2 prevalence in this community compared to a point prevalence estimate.

In this study, it is demonstrated how neutralizing assays serve as a check on the accuracy of SARS-CoV-2 antibody screening tests. Rapid, significant contributions by scientists worldwide have produced serologic SARS-CoV-2 data that has been consistent in one important way: variability. This study confirms this high variability in serologic assay performance and provides further data that no single serologic assay provides perfect prediction for viral neutralizing ability. In some scenarios, two-tiered testing would allow parsing of subjects into groups important for further study in the setting of large-scale vaccine trials (1) those with positive ELISA antibody detection and confirmed neutralization and (2) those with positive ELISA antibody detection but no evidence of neutralization. Group 1 would allow tracking of subjects with known neutralization titers for evidence of reinfection vs. possible protective immunity. Group 2 would allow study for increased identification of true false positives vs. individuals that do not develop functional and/or lasting neutralizing antibodies. Therefore, in some applications a two-tiered testing strategy in which a high-throughput pseudovirus assay is coupled to an accurate serologic assay would provide key data to identify subjects with possible protective immunity to SARS-CoV-2, and to assess vaccine efficacy. At the time of this study, sera from vaccine clinical trial studies were not available to be evaluated. However, mRNA vaccines against the spike protein have demonstrated detectable antibodies with RBD and spike IgG platforms similar to the platforms investigated here.

This study is believed to be the first to comprehensively assess serologic assay performance in an unknown cohort across antigen and antibody isotype comparisons in order to determine a practical and accurate method to determine the presence of SARS-CoV-2 neutralizing antibodies. By coupling ELISA with virus neutralization assessment, the accuracy of testing based was able to be determined on the key functional outcome of viral neutralization. Pseudoneutralization or FRNT assays provide a comparable test of neutralization to PRNTs and could be rapidly developed with a new spike IgG3 or an existing ELISA platform for two-tiered testing (see, e.g., Suthar M S, et al. Rapid generation of neutralizing antibody responses in COVID-19 patients. medRxiv 2020; Muruato A E, et al. A high-throughput neutralizing antibody assay for COVID-19 diagnosis and vaccine evaluation. bioRxiv 2020; and Crawford K H D, et al. Protocol and Reagents for Pseudotyping Lentiviral Particles with SARS-CoV-2 Spike Protein for Neutralization Assays. Viruses 2020; 12). This study provides compelling evidence that a two-tiered testing scheme ideally consisting of assessment of spike IgG3 antibodies and virus neutralization analysis optimally facilitates staging patients for SARS-CoV-2 antibody prevalence and a possible correlate of humoral immune protection to monitor clinical status and vaccine efficacy.

### Methods

Sample collection. Venipuncture collected 6-10 mls of blood in EDTA blood collection tubes and spun at 1000 rcf for 10 minutes. Plasma was separated, inactivated in a 56° C. water bath for 1 hour, and stored at −80 C.

ELISA. ELISA assays were performed as previously described (Stadlbauer D, et al. SARS-CoV-2 Seroconversion in Humans: A Detailed Protocol for a Serological Assay, Antigen Production, and Test Setup. Curr Protoc Microbiol 2020; 57:e100). Briefly, high-binding plates (ThermoScientific) were coated with SARS-CoV-2 RBD (Stadlbauer D, et al. SARS-CoV-2 Seroconversion in Humans: A Detailed Protocol for a Serological Assay, Antigen Production, and Test Setup. Curr Protoc Microbiol 2020; 57:e100), SARS-CoV-2 spike (Walls A C, et al. Structure, Function, and Antigenicity of the SARS-CoV-2 Spike Glycoprotein. Cell 2020; 181:281-92.e6), or UV-inactivated SARS-CoV-2 (WA1, BEI resources) and incubated overnight at 4° C. Plates were blocked in PBST+3% milk for lhr at RT. Three-fold serial dilutions of plasma were added to plates in biological duplicates. Samples and the positive control spike-binding antibody CR3022 (Abcam, ab273073) were included on plates with IgG antibody binding. Following 2 hr incubation and washes, anti-human secondary antibodies conjugated to HRP were diluted 1:3000 and added to plates: IgG (Thermofisher 31410), IgG1 (Southern Biotech 9054), IgG3 (Southern Biotech 9210), IgM (Sigma A6907), IgA (Sigma A0295). Following lhr incubation and washes, SigmaFast OPD was added to plates. Ten minutes later, 2M H2504 was added to wells stopping the reaction and plates read at an absorbance of 490 nm (BioTek Epoch). OD values for each sample dilution were plotted and area under the curve (AUC) was calculated using Prism. AUC analyses perform more accurately to inform outcomes compared to endpoint titer; the experiments were designed to apply AUC analyses (Chao C C, et al. An ELISA assay using a combination of recombinant proteins from multiple strains of Orientia tsutsugamushi offers an accurate diagnosis for scrub typhus. BMC Infect Dis 2017; 17:413; and Hartman H, et al. Absorbance summation: A novel approach for analyzing high-throughput ELISA data in the absence of a standard. PLoS One 2018; 13:e0198528).

Plaque reduction neutralization test (PRNT). PRNT analyses were performed as previously described (Erasmus J H, et al. Single-dose replicating RNA vaccine induces neutralizing antibodies against SARS-CoV-2 in nonhuman primates. bioRxiv 2020). Briefly, four-fold serial dilutions of heat inactivated plasma was mixed 1:1 with 600 PFU/ml SARS-CoV-2 WA-1 (BEI resources) in DPBS (Fisher Scientific)+0.3% cold water fish skin gelatin (Sigma G7041) and incubated for 30 min at 37 degrees. The virus plasma mixture was added in duplicate, along with virus and mock controls, to Vero cells (ATCC) in 12-well plates and incubated for lhr at 37 degrees. Following adsorption, plates were washed with DPBS and overlayed with a 1:1 mixture of 2.4% Avicel RC-591 (FMC)+2×MEM (ThermoFisher) supplemented with 4% heat-inactivated FBS and Penicillin/Streptomycin (Fisher Scientific). Plates were incubated for 2 days at 37 degrees. Overlay was removed and plates were washed with DPBS and fixed in 10% formaldehyde (Sigma-Aldrich) in DPBS for 30 minutes at room temperature. Plates were washed again with DPBS and stained with 1% crystal violet (Sigma-Aldrich) in 20% EtOH (Fisher Scientific). Plaques were enumerated and percent neutralization was calculated as 100 minus the number of plaques in serum+virus dilution wells divided by the number of plaques in virus only control wells times 100. PRNT₅₀ and PRNT₈₀ values are shown as inverse serum dilution and were determined by calculating the 50% and 80% sigmoidal interpolation of the percent neutralization of the samples in Prism. R² values were determined using a nonlinear regression fit in Prism.

University of Washington NP Assay. Plasma samples were run on the Abbott Architect instrument following the Abbott SARS-CoV-2 IgG assay instructions. Qualitative results and index values reported by the instrument were used in analysis. Values>1=1.4 were considered a positive result.

Statistics. Age and sex distributions of the Group B subjects were compared to the population of the greater Seattle area from U.S. Census estimates. Pearson's chi-square test was used for sex comparisons and the Kolmogorov-Smirnov (KS) test for age comparisons. Weights were constructed for over and under sampling for age and sex by taking the ratio of the proportion of the Seattle Census versus the Group B proportion. A prevalence estimate was calculated with weight adjustments (see Supplemental Data, below). Fisher's exact test was used to compare the rate of positive neutralizing antibodies against SARS-CoV-2 between exposed and unexposed groups. The values for sensitivity, specificity, PPV, and NPV for the ELISA platforms and PRNT were calculated using Prism software. P-values were calculated with Fisher's exact test. The 95% confidence intervals were calculated using the hybrid Wilson/Brown method.

Human participants. This study was conducted under University of Washington institutional review board number 000098108. The study protocol was reviewed and approved prior to enrollment of any subjects. Subjects were provided with information about the study, risks associated, and how their privacy would be protected. To enroll in the study each subject provided verbal understanding and written consent.

### Supplemental Data

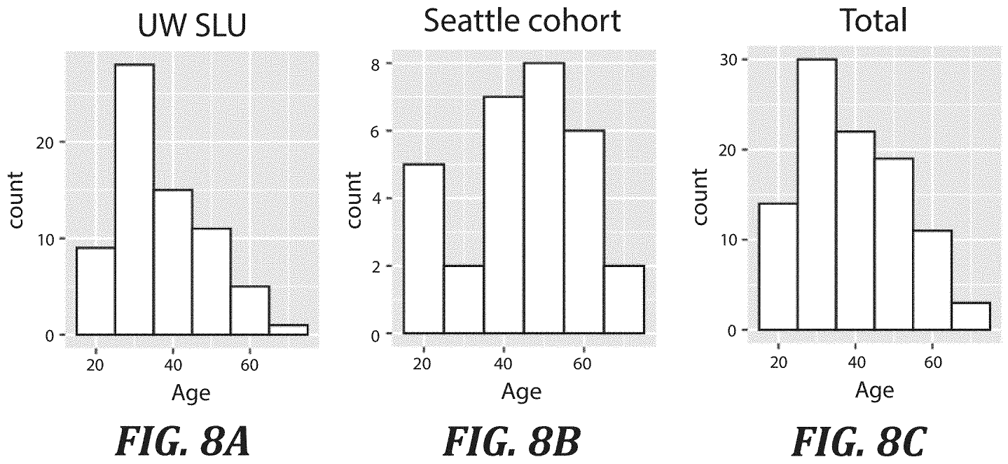

Plot the distribution for age and sex. The histograms for age for both groups and the total are shown in FIGS. 8A-8C. A summary of the sex distribution is in TABLE 5.

TABLE 5
Summary of male and female representation
in the two surveyed cohorts.

| | Male | Female |
|---|---|---|
| UW SLU | 29 | 40 |
| Seattle Cohort | 9 | 22 |
| Total | 38 | 62 |

For sex, the Pearson's chi-square test was used, which compares the proportions of male/female in two groups—the random samples and the SLU employees. Since we have a sample size of at least 30 in each group with at least 5 males and females in each group, and sex is a categorical variable, this test should give a highly accurate statement (a p-value) of the significance of the observed data. The test gives a p-value of 0.017.

For age, since the census data group age into different categories, only the distribution of age can be tested in those groups. The sample were broken into corresponding groups by their age: age less than 5, from 5 to 17, from 18 to 24, etc. Since there are many categories, the Kolmogorov-Smirnov (KS) test was used, which is a test for detecting differences between the distributions in two samples. The test gives a p-value of 0.008.

These p-values indicate that the distribution of age and sex for the sample is statistically significantly different from the Seattle population

Weighted Logistic Regression.

Based on the results of previous analyses, a weighted logistic regression model was used to estimate the overall Seattle prevalence. The weights account for oversampling and under-sampling certain groups of people. The weights were constructed by taking the ratio of the population proportion versus the sample proportion. In this implementation, the sample proportion was first computed by dividing the counts by the sample size, and then constructing weights based on the method described above. As for implementing the logistic regression model, the glm function in R was used, which enables use of weights.

The coefficients with corresponding p-values for the logistic regression model are shown in TABLE 6.

TABLE 6
Coefficients and p-values.

| | Estimate | Std. Error | z value | Pr(>\|z\|) |
|---|---|---|---|---|
| (Intercept) | −3.313515 | 0.4445265 | −7.454034 | 0 |

Using these estimates, an estimate of the prevalence and the confidence interval shown in TABLE 7 can be constructed.

TABLE7
Prevalence and confidence interval.

| | x |
|---|---|
| | 0.0351104 |
| 2.5% | 0.0130948 |
| 97.5% | 0.0728218 |

Fisher Exact Test for Difference in Rates

It was desired to test for the difference in positive rates between the group with positive exposure and the group without positive exposure. Given the presence of a small number of test positives in the sample without positive exposure, and the small sample size for the sample with positive exposure, the Fisher exact test we used. This test also assesses the difference between two groups, but is suitable for small sample sizes. The p-value is less than 1 e-4, so there is a statistically significant difference

Code for statistical analysis is set forth in Provisional Application No. 63/129,704, filed Dec. 23, 2020, incorporated herein by reference in its entirety.

While illustrative embodiments have been illustrated and described, it will be appreciated that various changes can be made therein without departing from the spirit and scope of the invention.

1. A method of detecting antibodies in a biological sample, wherein the antibodies are effective to neutralize a virus, the method comprising:
contacting the biological sample to a virus antigen, and
detecting the binding of one or more IgG3 antibodies to the antigen;
wherein binding of one or more IgG3 antibodies to the antigen indicates neutralizing antibodies in the biological sample.

2. The method of claim 1, wherein the virus is characterized as a respiratory virus for a subject.

3. The method of claim 1, wherein the virus is a coronavirus.

4. The method of claim 3, wherein the coronavirus causes the common cold, Middle East respiratory syndrome (MERS-CoV), severe acute respiratory syndrome (SARS), or coronavirus disease 2019 (COVID-19).

5. The method of claim 4, wherein the coronavirus is severe acute respiratory syndrome coronavirus 2 (SARS-CoV-2).

6. The method of claim 5, wherein the virus antigen is SARS-CoV-2 spike protein, or an immunogenic portion thereof.

7. The method of claim 1, wherein the biological sample is obtained from a subject.

8. The method of claim 7, wherein the presence of IgG3 antibodies indicates the subject has neutralizing antibodies against the virus.

9. The method of claim 1, wherein the virus antigen is immobilized on a solid substrate.

10. The method of claim 9, wherein detecting the binding of one or more IgG3 antibodies to the antigen comprises contacting the biological sample with an affinity reagent specific for IgG3 antibody.

11. The method of claim 10, wherein the affinity reagent is coupled directly or indirectly to a detectable moiety.

12. The method of claim 1, wherein the contacting and detecting are performed in an ELISA format or lateral flow format.

13. The method of claim 1, further comprising quantifying the IgG3 antibodies that bind to the antigen to provide an IgG3 level, and comparing the IgG3 level to a reference standard.

14. The method of claim 1, wherein the biological sample is or comprises blood, serum, plasma, sputum, nasopharyngeal swab specimen, buccal or oral swab specimen, cerebral spinal fluid, rectal swab or stool specimen, or is derived therefrom.

15. A method of determining whether a subject has neutralizing antibodies to a virus of the subfamily Orthocoronavirinae, the method comprising:
contacting a sample obtained from the subject with a virus antigen; and
detecting binding of one or more IgG3 antibodies to the virus antigen;
wherein detection of binding one or more IgG3 antibodies to the virus antigen indicates that the subject has neutralizing antibodies to the virus, and wherein lack of detection of binding one or more IgG3 antibodies to the virus antigen indicates that the subject lacks neutralizing antibodies to the virus.

16. The method of claim 15, wherein the virus antigen is SARS-CoV-2 spike protein, or a portion thereof.

17. The method of claim 15, wherein the virus antigen is immobilized on a solid substrate, wherein detecting the binding of one or more IgG3 antibodies to the antigen comprises contacting the sample with an affinity reagent specific for IgG3 antibody, and wherein the affinity reagent is coupled, directly or indirectly, to a detectable moiety.

18. The method of claim 15, wherein the presence of neutralizing antibodies indicates that the subject has been infected with or immunized against the virus resulting in at least partial protection against disease caused by the virus, and wherein the lack of neutralizing antibodies or lack of sufficient levels of neutralizing antibodies compared to a reference standard indicates that the subject has insufficient immunity against the virus and the method further comprises administering a vaccine or vaccine booster against the virus.

19. A kit comprising:
a virus antigen; and
an affinity reagent that specifically binds to IgG3 antibody.

20. The kit of claim 19, wherein the coronavirus is severe acute respiratory syndrome coronavirus 2 (SARS-CoV-2).


SEQUENCE LISTING (5 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKSNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLIIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFIVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPVQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK